(19) United States
(12) Patent Application Publication
(10) Pub. No.: US 2018/0119210 A1
(43) Pub. Date: May 3, 2018

(54) FETAL HAPLOTYPE IDENTIFICATION

(71) Applicant: SHAARE ZEDEK SCIENTIFIC LTD., Jerusalem (IL)

(72) Inventors: David A. ZEEVI, Jerusalem (IL); Gheona ALTARESCU, Jerusalem (IL)

(21) Appl. No.: 15/529,151

(22) PCT Filed: Nov. 24, 2015
(86) PCT No.: PCT/IL2015/051142

§ 371 Date: May 24, 2017

Related U.S. Application Data
(60) Provisional application No. 62/083,595, filed on Nov. 24, 2014.
(60) Provisional application No. 62/109,407, filed on Jan. 29, 2015.
(60) Provisional application No. 62/208,935, filed on Aug. 24, 2015.

(51) Int. Cl. C12Q 1/6827; C12Q 1/6883

(52) U.S. Cl. CPC C12Q 1/6827; C12Q 1/6883; C12Q 2535/122; C12Q 2600/172; C12Q 2600/156

(57) ABSTRACT

Methods and kits for prenatal genetic testing and particularly for identifying and/or analyzing fetal haplotype with a high degree of confidence are provided.

### FIELD OF INVENTION

The present invention is directed to; inter alia, methods and kits for prenatal genetic testing and particularly for identifying and/or analyzing fetal haplotype with a high degree of confidence.

### BACKGROUND OF THE INVENTION

Noninvasive prenatal genetic testing (NIPT) of whole chromosomal aneuploidies has already altered the landscape of prenatal diagnostics in the United States and increasingly worldwide. Aside from the noninvasiveness, advantages of NIPT include rapid turnaround, relatively low cost, and no hassle care for pregnant couples. Arguably, these benefits are largely made possible because it is not necessary to construct parental haplotypes in order to accurately diagnose chromosomal copy number. For noninvasive prenatal diagnosis (NIPD) of monogenic disease, on the other hand, this is not the case. In order for NIPD to take hold in the clinical setting it will be necessary to develop universal methodologies that apply to the diagnosis of any mutation, maternal or paternal, regardless of inheritance. Although some universal techniques for NIPD have already been described, each one requires time-consuming and sophisticated parental haplotype construction in advance of test interpretation (Fan eat al. 2012 Nature 487:320-324; Kitzman et al. 2012. Sci Transl Med 4:137ra176; and Lo et al. Sci Transl Med 2:61ra91).

The classic haplotype construction methodology is simpler to implement because it involves the collection of DNA samples from several family members for linkage analysis. Nevertheless, this process is often complicated or sometimes made impossible by low compliance, couple privacy concerns, or the unavailability of living first degree relatives. To address these issues, researchers have also developed various molecular and statistical techniques for family-independent haplotyping (Browning and Browning, 2011. Nat Rev Genet 12:703-714). Unfortunately, the described molecular techniques are either too expensive, too time-consuming, and/or too labor intensive for use in a clinical setting. Moreover, statistical approaches, which rely on high throughput analysis of population data, are not appropriate for clinical application.

Medical centers around the world offer invasive prenatal diagnostic services for local population-specific founder mutations on a routine basis. Depending on the carrier frequency within the population, founder mutation tests often comprise a significant component of the overall molecular testing in such healthcare laboratories. Some examples of common founder mutations for which prenatal testing would be relevant include those implicated in long QT syndrome within the Finnish population (Marj amaa et al. 2009 Ann Med 41:2.34-240); the delF508 mutation in CFTR causing cystic fibrosis in the caucasian European population (Moral et al. 1994, Nat Genet 7:169-175); a mutation in the SERPINA1 gene causing alpha1-antitrypsin deficiency in Scandinavian Caucasians (Cox et al. 1985, Nature 316:79-81); a mutation in Columbians causing early onset Alzheimer's disease (Lalli et al., 2013, Alzheimers Dement, S277-S283); and scores of founder mutations in the Tunisian (Romdhane et al., 2012 Orphanet J Rare Dis 7:52) and Ashkenazi Jewish (AJ) populations (Zlotogora, J. 2014, Mendelian disorders among Jews).

There is an unmet need for a rapid, cost-effective, and routine test that can be implemented for highly accurate fetal haplotype identification, such as for NIDD of monogenic disorders, without reliance on blood sample collection from relatives of the pregnant couple.

### SUMMARY OF THE INVENTION

The present invention provides, in some embodiments, methods and kits for identifying and/or analyzing fetal haplotype with a high degree of confidence.

According to another embodiment, the present invention provides a method for non-invasively predicting an increased risk of maternal and/or paternal haplotypes inherited by a fetus of a pregnant female, the method comprising:
(i) obtaining at least a replicate of a fetal nucleic acid sequence sequenced at a depth of at least 100×coverage, said fetal nucleic acid sequence being derived from DNA samples obtained from the pregnant female; and
(ii) analyzing said replicate of fetal nucleic acid sequence, wherein a high identity of said fetal haplotype to a consensus haplotype indicates that said fetus is a carrier of a maternal and/or paternal haplotype;
   thereby predicting an increased risk of a maternal and/or paternal haplotype inherited by said fetus.

According to another embodiment, the present invention provides a method for non-invasively predicting an increased risk of a monogenic disease or disorder in a fetus of a pregnant female, the method comprising:
(i) obtaining at least a replicate of a fetal nucleic acid sequence sequenced at a depth of at least 100×coverage, said fetal nucleic acid sequence being derived from DNA samples obtained from the pregnant female; and
(ii) analyzing said replicate of fetal nucleic acid sequence, wherein a high identity of said fetal haplotype to a consensus haplotype indicates that said fetus is a carrier of a maternal and/or paternal haplotype;
   thereby predicting an increased risk of a monogenic disease or disorder in said fetus.

According to some embodiments, said sample is a plasma sample. According to some embodiments, said DNA is plasma DNA. According to some embodiments, said plasma DNA is cell-free fetal DNA (cffDNA).

In another embodiment, said replicate of a fetal nucleic acid sequence is sequenced at a depth of at least 1,500×coverage. In another embodiment, said replicate of a fetal nucleic acid sequence is sequenced at a depth of at least 2,000×coverage. According to another embodiment, said fetal nucleic acid sequence is sequenced at a depth of at least 2,500×mean coverage. According another embodiment, said fetal nucleic acid sequence is sequenced at a depth of at least 3,000×mean coverage.

According to another embodiment, said analyzing said fetal nucleic acid sequence comprises comparing said fetal haplotype to a consensus haplotype. According to another embodiment, said consensus haplotype is a population-based haplotype based on subjects unrelated to said fetus.

According to another embodiment, said analyzing said replicate of fetal nucleic acid sequence comprises determining one or more paternal haplotype informative single-nucleotide polymorphism (SNP)s in at least one replicate of fetal nucleic acid, said paternal haplotype informative SNPs are not present in the maternal genotype, thereby determining unique paternal SNPs identified in the fetus.

According to another embodiment, said analyzing said replicate of fetal nucleic acid sequence comprises determining maternal haplotype informative SNPs in fetal nucleic acid, thereby determining maternal haplotype in said fetus.

According to another embodiment, said maternal haplotype comprises a founder haplotype encompassing a founder mutation, said method being useful for predicting an increased risk of said founder mutation in said fetus. According to another embodiment, said monogenic disease or disorder is caused by, or strongly associated with, a founder mutation. According another embodiment, said monogenic disease or disorder presents with autosomal recessive inheritance.

According to another embodiment, the present invention provides a kit for identifying or analyzing fetal haplotype with a high degree of confidence.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows pedigrees of glucosidase, beta, acid (GBA) mutation carrier families in the study presented herein. Mutations in GBA are indicated. Individuals with unknown genotypes at sample collection are shaded in gray. “WT” denotes a (wild-type) WT GBA allele; “wk” denotes the week of gestation at which maternal plasma was collected.

FIGS. 2A-C are illustrations of fine mapping of the consensus AJ N370S founder haplotype region. Hundreds of GBA-flanking SNPs (±250 kb from GBA) were sequenced in order to identify a conserved N370S founder haplotype. (2A) NGS-based homozygosity mapping with 7 unrelated homozygote N370S Gaucher patients (denoted as H1-H7) (14 N370S chromosomes) was used to identify a preliminary founder haplotype. (2B) A representative linkage-based inference of a familial N370S haplotype (hapN370S). This linkage analysis was performed for 6 different heteroallelic GBA N370S mutation carrier duos (6 N370S chromosomes from 6 sets of 2 first-degree family members carrying the N370S mutation). The resultant alleles were each compared separately to the haplotype from FIG. 2A until a consensus N370S haplotype was demarcated with a 5′ cutoff. (2C) Ultimately, the consensus AJ N370S founder haplotype (composed of 153 SNPs) used for NIPD was constructed from 20 different AJ N370S chromosome sequences. Notably, this analysis set a 5′ cutoff for the conserved N370S haplotype, but a 3′ cutoff could not be established. WT denotes a WT allele.

FIGS. 3A-D are illustrations depicting the immediate GBA-proximal locus and SNPs that were deep sequenced for the construction and typing of fetal alleles (as indicated in the “Haplotype legend”). (3A) In family 1, the paternal WT allele was diagnosed by inference from the family-based N370S-linked haplotype (squares). The consensus N370S haplotype could not be used to phase the paternal allele in the fetus due to paternal homozygosity in the founder haplotype region. (3B) On the other hand, the maternal N370S allele in family 1 was readily identified (in multiple sites) via the consensus haplotype, and this result was corroborated by equivalent matches to the family-based maternal N370S haplotype. (3C) For the family 2 maternal allele, the fetal N370S haplotype could only be matched to a single polymorphic site in the family-based haplotype (square). This site and 2 other fetal SNPs were definitively matched to the N370S mutation by comparison to the founder N370S haplotype. Therefore, in this case, it would not have been possible to reliably diagnose the maternal allele in the fetus without the N370S founder sequence. (3D) Haplotype legend for FIG. 3A-C.

FIGS. 4A-E are illustrations depicting the GBA locus (±2 Mb) and thousands of SNPs that were deep sequenced for the construction and typing of fetal alleles according to the analytical pipeline (as indicated in the key in 4E). (4A) An extended deep-sequencing panel was used to better fine map the conserved N370S founder haplotype, as in FIGS. 2A-C. Accordingly, a 301-SNP haplotype (termed “full-consensus N370S haplotype”) was identified in all N370S chromosomes in this study (28 chromosomes altogether). In addition, the consensus haplotype was found to extend 500 kb further downstream of GBA (620 additional SNPs) in 15 of 16 chromosomes from 8 N370S homozygotes. Furthermore, in all N370S homozygotes (but not all N370S carriers), the consensus haplotype was found to extend another 120 kb upstream of GBA (100 additional SNPs). Altogether, these extended haplotypes were termed “near-consensus N370S haplotypes.” (4B) The N370S haplotype from each N370S carrier parent in the study was carefully mapped according to homozygous regions and family-based linkage analysis. After comparison to the near-consensus haplotype in FIG. 4A, new parent-specific 5′ and/or 3′ demarcations of the N370S near-consensus haplotype were set (this haplotype was termed the “parent-specific consensus N370S haplotype”). (4C) In this example, deep sequencing of the GBA-flanking region in a fetus identified stretches of a linkage-based parental N370S haplotype that resided outside of the consensus N370S region. In addition, some stretches of fetal sequence could not be phased according to family-based linkage. (4D) When unphased fetal sequence, such as in FIG. 4C, fell within the parent-specific consensus N370S haplotype (as determined in FIG. 4B), the consensus information was used to phase the fetus (here, with the N370S-linked haplotype), thereby increasing confidence in the diagnostic test result.

FIGS. 5A-K are illustrations depicting the GBA locus (±2 Mb) and SNPs that were deep sequenced for the construction and typing of fetal alleles (as indicated in the “Haplotype legend” in (5K). The numbers shown under DFM denote the distance from mutation (in Mb). The noninvasively identified fetal alleles were: (5A) WT paternal; (5B) N370S maternal; (5C) N370S maternal; (5D) N370S maternal; (5E) WT paternal; (5F) WT maternal; (5G) N370S paternal; (5H) N370S paternal; (5I) L444P (non-N370S) maternal, and (5J) 84GG (non-N370S) maternal. Note the utility of the N370S consensus haplotype for fetal typing in FIGS. 5B, 5C, and 5H. The near-consensus N370S haplotype also aided fetal typing in FIGS. 5B, 5C, 5F-H, and 5J.

FIGS. 6A-D are tables listing a consensus Ashkenazi Jewish N370S founder haplotype. The following abbreviations were used: Ch, chromosome; REF, reference nucleotide (dbSNP Build 138); ALT, alternate (non-reference) nucleotide (dbSNP Build 138). For the consensus AJ N370S haplotype: “A”=dbSNP reference nucleotide; “B”=dbSNP non-reference nucleotide. The region shaded in gray indicates GBA gene 5′ and 3′ locus boundaries.

FIG. 7 is a table listing the identification of the paternal allele in the family 1 fetus (small panel). The following abbreviations were used: “Ch” chromosome; “DFM” distance from mutation; “PGT” paternal genotype; “MGT” maternal genotype; “FL” fetal load; “rep” replicate plasma DNA sample, “RD” sequencing read depth; “BAF”, B-allele frequency; “PHiF” paternal haplotype in fetus; “PFB N370S” paternal family-based N370S-linked haplotype; “FAI” fetal allele identity; “DPAiF” diagnosed paternal allele in fetus. dbSNP ID or GBA mutation are marked by underlined lettering. For parental genotypes “AA”=homozygote dbSNP reference allele; “BB”=homozygote dbSNP non-reference allele; “AB”=heterozygote. Fetal load is 2×(mean paternal fetal fraction) as determined from SNP I and/or SNP II data (see methods section). B-allele frequency (BAF) is the % frequency of (B-allele reads)/(total read depth (RD)) at the indicated nucleotide position; bold BAF data was used to construct “PHiF”. The paternal fetal haplotype (PHiF) was determined from SNP II data (as described in the methods section); the paternal N370S-linked haplotype (PFB N370S) was determined from family-based linkage analysis; the N370S consensus haplotype (N370S cons) was derived according to FIG. 2. An “-” indicates that no haplotype data was available at the given position. Bold alleles were used for diagnosis of the paternal allele in the fetus (“DPAiF”). Fetal allele identity (FAI) was determined by comparing the “PHiF” haplotype to the “PFB N370S” haplotype

FIG. 8 is a table presenting a preliminary summary of noninvasive prenatal diagnosis with validation. “N/A”| depicts not applicable due to paternal homozygosity in consensus N370S haplotype region.

FIG. 9 is a table summarizing the identification of the maternal allele in the family 1 fetus (small panel). The following abbreviations were used: “MHiF” maternal haplotype in fetus; “MFB N370S” maternal family-based N370S-linked haplotype; “N370S cons” consensus N370S haplotype, “FAI” fetal allele identity; “DMAiF” diagnosed maternal allele in fetus; dbSNP ID or GBA mutation (underlined lettering); For parental genotypes “AA”=homozygote dbSNP reference allele; “BB”=homozygote dbSNP non-reference allele; “AB”=heterozygote; Fetal load is 2×(mean paternal fetal fraction) as determined from SNP I and/or SNP II data (see methods section); B-allele frequency is the % frequency of (B-allele reads)/(total read depth (RD)) at the indicated nucleotide position; other abbreviations are the same as in FIG. 7. The maternal fetal haplotype (MHiF) was determined from SNP III data (as described in the methods section); the maternal N370S-linked haplotype (MFB N370S) was determined from family-based linkage analysis; the N370S consensus haplotype (N370S cons) was derived according to FIG. 2. An “-” indicates that no haplotype data was available at the given position. Bold alleles were used for diagnosis of the maternal allele in the fetus (“DMAiF”). Fetal allele identity (FAI) was determined by comparing the “MHiF” haplotype to either the “MFB N370S” and/or “N370S cons” haplotypes.

FIG. 10 is a table summarizing the identification of the maternal allele in the family 2 fetus (small panel). Abbreviations and definitions are the same as in FIG. 9. The GBA mutation is marked by underlined lettering.

FIGS. 11A-G are tables listing a consensus Ashkenazi Jewish N370S founder haplotype. The following abbreviations were used: “Ch” chromosome; “REF” reference nucleotide (dbSNP Build 141); “ALT” alternate (non-reference) nucleotide (dbSNP Build 141). For the consensus AJ N370S haplotype: “A”=dbSNP reference nucleotide; “B”=dbSNP non-reference nucleotide. The region shaded in gray indicates GBA intragenic loci.

FIG. 12 is a table listing parental family-based haplotype information (from large sequencing panel). The following abbreviations were used: “WT” wild type; “N/A” not applicable.

FIG. 13 is a table summarizing the identification of the paternal allele in the family 1 fetus (large panel). Abbreviations and definitions are the same as in FIG. 7. GBA mutation is marked by underlined lettering.

FIG. 14 is a table summarizing the identification of the maternal allele in the family 1 fetus (large panel). Abbreviations and definitions are the same as in FIG. 9 apart from the consensus N370S haplotype which is determined according to FIG. 4. The GBA mutation is marked by underlined lettering.

FIG. 15 is a table summarizing the identification of the maternal allele in the family 2 fetus (large panel). Abbreviations and descriptions are the same as in FIG. 12.

FIG. 16 is a table summarizing the identification of the maternal allele in the family 3 fetus (large panel). Abbreviations and definitions are the same as in FIG. 14 with the following modifications: E—the maternal fetal haplotype (MHiF) was determined from SNP III data (as described in the methods section); the maternal N370S-linked (MFB N370S) and maternal V394L-linked (MFB V394L) haplotypes were determined by family-based linkage analysis; the N370S consensus haplotype (N370S cons) was derived according to FIG. 2. An “-” indicates that no haplotype data was available at the given position. Bold alleles were used for diagnosis of the maternal allele in the fetus (“DMAiF”). F— fetal allele identity (FAI) was determined by comparing the “MHiF” haplotype to the “MFB N370S”, “MFB V394L”, and/or “N370S cons” haplotypes.

FIG. 17 is a table summarizing the identification of the paternal allele in the family 4 fetus (large panel). Abbreviations and definitions are the same as in FIG. 7 with the following modifications: E—the paternal fetal haplotype (PHiF) was determined from SNP II data (as described in the methods section); the paternal R496H-linked (PFB R496H) and wild type-linked (PFB WT) haplotypes were determined from family-based linkage analysis. An “-” indicates that no haplotype data was available at the given position. Bold alleles were used for diagnosis of the paternal allele in the fetus (“DPAiF”). F— fetal allele identity (FAI) was determined by comparing the “PHiF” haplotype to the “PFB R496H” and/or “PFB WT” haplotypes.

FIGS. 18 A-B are tables summarizing the identification of the maternal allele in the family 4 fetus (large panel). Abbreviations and definitions are the same as in FIG. 14 with the following modifications: E—the maternal fetal haplotype (MHiF) was determined from SNP III data (as described in the methods section); the maternal N370S-linked (MFB N370S) and maternal wild type-linked (MFB WT) haplotypes were determined by family-based linkage analysis; the N370S consensus haplotype (N370S cons) was derived according to FIG. 2. An “-” indicates that no haplotype data was available at the given position. Bold alleles were used for diagnosis of the maternal allele in the fetus (“DMAiF”). F— fetal allele identity (FAI) was determined by comparing the “MHiF” haplotype to the “MFB N370S”, “MFB WT”, and/or “N370S cons” haplotypes.

FIGS. 19A-B is a table summarizing the identification of the paternal allele in the family 5 fetus (large panel). Abbreviations and definitions are the same as in FIG. 7 with the following modifications: E—the paternal fetal haplotype (PHiF) was determined from SNP III data (as described in the methods section); the paternal N370S-linked (PFB N370S) and paternal del55-linked (PFB del55) haplotypes were determined by family-based linkage analysis; the N370S consensus haplotype (N370S cons) was derived according to FIG. 2. An “-” indicates that no haplotype data was available at the given position. Bold alleles were used for diagnosis of the paternal allele in the fetus (“DPAiF”). F— Fetal allele identity (FAI) was determined by comparing the “PHiF” haplotype to the “PFB N370S”, “PFB del55”, and/or “N370S cons” haplotypes. G-near consensus N370S haplotype as determined according to FIG. 4.

FIG. 20 is a table summarizing the identification of the paternal allele in the family 6 fetus (large panel). Abbreviations and definitions are the same as in FIG. 7 with the following the following modification: G-near consensus N370S haplotype as determined according to FIG. 4.

FIG. 21 is a table summarizing the identification of the maternal allele in the family 7 fetus (large panel). Abbreviations and definitions are the same as in FIG. 14 with the following modifications: E—the maternal fetal haplotype (MHiF) was determined from SNP III data (as described in the methods section); the maternal N370S-linked (MFB N370S) and maternal L444P-linked (MFB L444P) haplotypes were determined by family-based linkage analysis; the N370S consensus haplotype (N370S cons) was derived according to FIG. 2. An “-” indicates that no haplotype data was available at the given position. Bold alleles were used for diagnosis of the maternal allele in the fetus (“DMAiF”). F-fetal allele identity (FAI) was determined by comparing the “MHiF” haplotype to the “MFB N370S”, “MFB L444P”, and/or “N370S cons” haplotypes.

FIG. 22 is a table summarizing the identification of the maternal allele in the family 8 fetus (large panel). Abbreviations and definitions are the same as in FIG. 14 with the following modifications: E—the maternal fetal haplotype (MHiF) was determined from SNP III data (as described in the methods section); the maternal N370S-linked (MFB N370S) and maternal 84GG-linked (MFB 84GG) haplotypes were determined by family-based linkage analysis; the N370S consensus haplotype (N370S cons) was derived according to FIG. 2. An “-” indicates that no haplotype data was available at the given position. Bold alleles were used for diagnosis of the maternal allele in the fetus (“DMAiF”). F-Fetal allele identity (FAI) was determined by comparing the “MHiF” haplotype to the “MFB N370S”, “MFB 84GG”, and/or “N370S cons” haplotypes

FIG. 23 is a table presenting a summary of noninvasive prenatal diagnosis (using large sequencing panel) with validation. The following abbreviations were used: A—Due to N370S carrier homozygosity in consensus N370S haplotype region; B— V394L is denoted p.V433L (c. 1297G>T) according to GenBank accession: NM_001005741.2; C— R496H is denoted p.R535H (c. 1604G>A) according to GenBank accession: NM_001005741.2; D—del55 is denoted c. 1263_1317del55 according to GenBank accession: NM_001005741.2 and E—L444P is denoted p.L483P(c. 1448T>C) according to GenBank accession: NM_001005741.2.

### DETAILED DESCRIPTION OF THE INVENTION

The present invention provides, in some embodiments, methods and kits for identifying and/or analyzing fetal haplotype with a high degree of confidence.

By virtue of identifying fetal haplotype, the invention may be applicable for many methods, including but not limited to, noninvasive prenatal diagnosis (NIPD), such as, of a monogenic disease, or alternatively, for human leukocyte antigen (HLA) typing, such as, for screening potential cord blood donors.

The present invention is based, in part, on the understanding that the common denominator among all population-specific mutations is that they each appear with their own mutation-flanking molecular fingerprint or haplotype. In particular embodiments of the invention, this fingerprint is used as a tool for fetal haplotype identification such as for NIPD. Thus, by means of highly targeted next generation sequencing (NGS), it is exemplified herein that fine-mapping of a founder mutation fingerprint is a potentially valuable asset for NIPD of an autosomal recessive disease. According to advantageous embodiments, the methods described herein alleviate the hassle of constructing family-specific haplotypes (e.g., for founder mutation NIPD). Moreover, the use of mutation-specific fingerprints eliminates the need for sophisticated molecular haplotyping methods, thereby effecting major savings with regard to test duration, reagent cost, and labor expenditure.

Parental haplotype construction is a primary drawback to NIPD of monogenic disease. Family-specific haplotype assembly is typically necessary for diagnosis of minuscule amounts of circulating cell-free fetal DNA. Nevertheless, this endeavor still hampers practical application of NIPD in the clinic because current haplotyping techniques are still too time-consuming and laborious to be carried out within the limited time constraints of prenatal testing.

To address this pitfall, the inventors have devised a universal strategy for rapid fetal haplotype identification, thereby being useful for NIPD of a prevalent mutation. Accordingly, some embodiments of the invention are applicable in the context of NIPD, including but not limited to, of a monogenic disease, and particularly of diseases associated with autosomal recessive disease-causing mutations.

As exemplified herein below using a non-limiting founder mutation, a consensus Gaucher disease-associated mutation-flanking haplotype was fine-mapped by means of targeted next generation sequencing, so as to successfully diagnose seven unrelated fetuses. One skilled in the art will appreciate that the methods described herein are shown as a non-limiting demonstration for accurate fetal haplotype identification. Accordingly, the methods and kits of the invention may be used for NIPD of any worldwide autosomal recessive founder mutation.

In additional embodiments, the disclosed invention is applicable for human leukocyte antigen (HLA) typing of a fetus, including but not limited to, for screening potential cord blood donors.

Thus, the present invention provides rapid, economical, and readily adaptable methods and kits for highly accurate fetal haplotype identification.

According to some embodiments, there is provided a method for predicting an increased risk of maternal and/or paternal haplotypes inherited by a fetus of a pregnant female.

According to some embodiments, said method comprises obtaining or providing a sample obtained from a pregnant female, referred to herein as “maternal sample”. In one embodiment, the maternal sample includes any processed or unprocessed, solid, semi-solid, or liquid biological sample, e.g., blood, urine, saliva, mucosal samples (such as samples from uterus or vagina, etc.). For example, the maternal sample may be a sample of whole blood, partially lysed whole blood, plasma, or partially processed whole blood.

According to some embodiments, said maternal blood sample is plasma DNA, e.g., cell-free fetal DNA (cffDNA) or free floating DNA from maternal whole blood.

The sample of maternal blood can be obtained by standard techniques, such as using a needle and syringe. In another embodiment, the maternal blood sample is a maternal peripheral blood sample. Alternatively, the maternal blood sample can be a fractionated portion of peripheral blood, such as a maternal plasma sample. In another embodiment, once the blood sample is obtained, total DNA can be extracted from the sample using standard techniques known to one skilled in the art. A non-limiting example for DNA extraction is the FlexiGene DNA kit (QIAGEN). In another embodiment, maternal plasma may be further separated from peripheral blood by centrifugation, such as exemplified herein, at 1,900×g for 10 minutes at 4° C. The plasma supernatant may be re-centrifuged at 16,000×g for 10 minutes at 4° C. In another embodiment, a fraction of the resulting supernatant is used for cell-free DNA extraction, to thereby receive maternal plasma DNA extracts. Standard techniques for receiving cell-free DNA extraction are known to a skilled artisan, a non-limiting example of which is the QIAamp Circulating Nucleic Acid kit (QIAGEN). In some embodiments, the total DNA is subsequently fragmented, such as to sizes of approximately 300 bp-800 bp. For example, the total DNA can be fragmented by sonication.

In some embodiments, the methods described herein include a step of determining the amount of fetal nucleic acid within the obtained DNA sample (e.g., concentration, relative amount, absolute amount, copy number, and the like).

In some cases, the amount of fetal nucleic acid in a sample is referred to as “fetal fraction”. In some embodiments, “fetal fraction” refers to the fraction of fetal nucleic acid in circulating cell-free nucleic acid in the maternal sample. A determinant of the resolution of the fetal genetic map or fetal genomic sequence at a given level, or depth, of DNA sequencing is the fractional concentration of fetal DNA in the maternal biological sample. Typically, the higher the fractional fetal DNA concentration, the higher is the resolution of the fetal genetic map or fetal genomic sequence that can be elucidated at a given level of DNA sequencing. As the fractional concentration of fetal DNA in maternal plasma is higher than that in maternal serum, maternal plasma is typically considered a more preferred maternal biological sample type than maternal serum.

A size fractionation step can also be performed on the nucleic acid molecules in the maternal sample. As fetal DNA is known to be shorter than maternal DNA in maternal plasma, the fraction of smaller molecular size can be harvested and then used for the methods of the invention. Such a fraction would contain a higher fractional concentration of fetal DNA than in the original biological sample.

Thus, the sequencing of a fraction enriched in fetal DNA can allow one to construct the fetal genetic map or deduce the fetal genomic sequence with a higher resolution at a particular level of analysis (e.g. depth of sequencing), than if a non-enriched sample has been used.

Typically, applying said size fractionation step may alter the technology more cost-effective. As non-limiting examples of methods for size fractionation, one could use (i) gel electrophoresis followed by the extraction of nucleic acid molecules from specific gel fractions; (ii) nucleic acid binding matrix with differential affinity for nucleic acid molecules of different sizes; or (iii) filtration systems with differential retention for nucleic acid molecules of different sizes.

In another embodiment, the maternal plasma DNA extracts are pre-amplified, in replicate (e.g., in duplicate or more), using standard techniques, a non-limiting example of which is the SurePlex Amplification System (BlueGnome). In particular embodiments, said pre-amplification step is performed ahead of downstream processing, i.e., before the analysis step. As exemplified herein, undertaking the methods of the invention using at least a replicate of amplified fetal nucleic acid sequences, substantially augmented statistical confidence in each individual fetal SNP genotype call.

In some embodiments of the method disclosed herein, the DNA is amplified (e.g., in replicate or more) after plasma DNA is extracted. As used herein, the term “amplified” is intended to mean that additional copies of the DNA are made to thereby increase the number of copies of the DNA, which is typically accomplished using the polymerase chain reaction (PCR). Additional methods of amplification are known to one skilled in the art.

In another embodiment, said replicate of a fetal nucleic acid sequence is sequenced by next generation sequencing (NGS). In another embodiment, said replicate of a fetal nucleic acid sequence is sequenced at a depth of at least 100× coverage, of at least 500× coverage, at least 1,000× coverage, of at least 1,500× coverage, of at least 2,000× coverage, of at least 2,500× coverage or of at least 3,000× coverage, as well as individual numbers within that range. Each possibility represents a separate embodiment of the invention.

As used herein, the term “depth” refers to the number of times a nucleotide is read during the sequencing process. The term “coverage” refers to the average number of reads representing a given nucleotide in the reconstructed sequence. Accordingly, deep sequencing indicates that the total number of reads is many times larger than the length of the sequence under study.

According to another embodiment, said analyzing said fetal nucleic acid sequence comprises comparing said fetal haplotype to a consensus haplotype. According another embodiment, said consensus haplotype is a population-based haplotype based on subjects unrelated to said fetus. In some embodiments, a consensus founder haplotype for a specific disease or condition is obtained from a publicly available haplotype database, such as but not limited to, HapMap or deCode.

The term “consensus haplotype” as used herein refers to a DNA sequence surrounding a specific genomic locus of interest, such as but not limited to, a founder mutation locus, an HLA locus or a genetic susceptibility locus. In some embodiments, the consensus haplotype may span upstream (+) or downstream (−) of the locus. In another embodiment, the consensus haplotype is both upstream and downstream of the locus of interest.

The required length of consensus haplotype for obtaining high accuracy predictions depends on a number of variables such as but not limited to, SNP frequency and recombination susceptibility of the target genomic region. According to some embodiments, the length of said consensus haplotype is of at least +/−250 kb from the locus of interest. According to some embodiments, the length of said consensus haplotype is of at least +/−500 kb from the locus of interest. According to some embodiments, the length of said consensus haplotype is of at least +/−1 Mb from the locus of interest. According to some embodiments, the length of said consensus haplotype is of at least +/−3 Mb from the locus of interest. According to some embodiments, the length of said consensus haplotype is of at least +/−5 Mb from the locus of interest.

The throughput of the above-mentioned sequencing-based methods can be increased with the use of indexing or barcoding. Thus, a sample or subject-specific index or barcode can be added to nucleic acid fragments in a particular nucleic acid sequencing library. Then, a number of such libraries, each with a sample or subject-specific index or barcode, are mixed together and sequenced together. Following the sequencing reactions, the sequencing data can be harvested from each sample or patient based on the barcode or index. This strategy can increase the throughput and thus the cost-effectiveness of embodiments of the current invention.

In one embodiment, the nucleic acid molecules in the biological sample can be selected or fractionated prior to quantitative genotyping (e.g. sequencing). In one variant, the nucleic acid molecules are treated with a device (e.g. a microarray) which can preferentially bind nucleic acid molecules from selected loci in the genome. Then, the sequencing can be performed preferentially on nucleic acid molecules captured by the device. This scheme will allow one to target the sequencing towards the genomic region of interest. In another embodiment, said sequencing is of loci comprising single nucleotide polymorphisms (SNPs), such as SNPs linked to a disease or disorder. One skilled in the art will appreciate that many SNPs are linked to a disease or disorder. In one embodiment, said SNP is linked to a founder mutation. In another embodiment, said sequencing is of founder mutation-flanking SNPs.

As used herein, “founder mutation” refers to a mutation that appears in the DNA of one or more individuals who are founders of a distinct population. Founder mutations can initiate with changes that occur in the DNA and are typically passed down to other generations.

In one embodiment, said disease is Gaucher, such as Gaucher type I. In another embodiment, said founder mutation is N370S (c. 1226A>G or p.N409S according to GenBank accession #: NM_001005741.2). In another embodiment, said founder mutation is 84GG (c. 84dupG on GenBank sequence NM_001005741.2). None limiting examples of founder mutations for which the prenatal testing of the invention would be relevant include those implicated in long QT syndrome within the Finnish population (Marjamaa et al. 2009 Ann Med 41:2.34-240); the delF508 mutation in CFTR causing cystic fibrosis in the caucasian European population (Moral et al. 1994, Nat Genet 7:169-175); a mutation in the SERPINA1 gene causing alpha1-antitrypsin deficiency in Scandinavian Caucasians (Cox et al. 1985, Nature 316:79-81); a mutation in Columbians causing early onset Alzheimer's disease (Lalli et al., 2013, Alzheimers Dement, S277-S283); and scores of founder mutations in the Tunisian (Romdhane et al., 2012 Orphanet J Rare Dis 7:52) and Ashkenazi Jewish (AJ) populations (Zlotogora, J. 2014, Mendelian disorders among Jews); mutations residing in the HBB gene which cause Beta-thalassemia in Mediterranean and Asian populations (Cao and Galanello. Genet Med. 2010 February; 12(2):61-76); the mutation c. 191dupA in the ANOS gene which is highly predictive of adult limb-girdle muscular dystrophy (Bushby et al. 2011, Brain January; 134(Pt 1):171-82).

Founder mutations have been also identified in many types of cancers. Some non-limiting examples of cancer related founder mutations are mutations in the BRCA1 and BRCA2 associated with breast cancer. The founder mutations P57T, R603C, Q630C and A628K variants of the netrin-1 receptor UNCSC have been implicated in the predisposition and carcinogenesis leading to solid cancers in humans (EP patent application 2267153).

In another embodiment, the methods and kits disclosed herein are useful for determining the susceptibility to a microdeletion or microduplication syndrome, such as Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome, or a disorder selected from the group consisting of Cri du Chat syndrome, Retinoblastoma, Wolf-Hirschhorn syndrome, Wilms tumor, spinobulbar muscular atrophy, cystic fibrosis, Gaucher disease, Marfan syndrome and sickle cell anemia.

One skilled in the art will appreciate that the length of sequence to be analyzed according to the methods described herein, depends on the specific haplotype to be determined. In some embodiments, a number of loci along a chromosome that needs to be sequenced is between 5,000 and 10,000 loci; between 10,000 and 50,000 loci; between 1,000 and 500 loci; between 500 and 300 loci; between 300 and 200 loci; between 200 and 150 loci; between 150 and 100 loci; between 100 and 50 loci; between 50 and 20 loci; or between 20 and 10 loci. In some embodiments, at least 2 loci, at least 10 loci, at least 20 loci, at least 50 loci, at least 100 loci, at least 1,000 loci, at least 5,000 loci or at least 10,000 are sequenced.

In another embodiment, the method further comprises analyzing said replicate of fetal nucleic acid sequence, wherein a high identity of said fetal haplotype to a consensus haplotype indicates that said fetus is a carrier of a maternal and/or paternal haplotype.

In some embodiments, the term “high identity” as used herein refers to at least 90% identity of said fetal haplotype to a consensus haplotype. In another embodiment high identity refers to at least 95% identity of said fetal haplotype to a consensus haplotype. In another embodiment high identity refers to at least 98% identity of said fetal haplotype to a consensus haplotype. In another embodiment high identity refers to at least 99% identity of said fetal haplotype to a consensus haplotype.

In another embodiment, the method further comprises analyzing said replicate of fetal nucleic acid sequence, wherein a high identity of said fetal haplotype to a family-based haplotype indicates that said fetus is a carrier of a maternal and/or paternal haplotype.

According to another embodiment, said analyzing said replicate of fetal nucleic acid sequence comprises determining one or more paternal haplotype informative single-nucleotide polymorphism (SNP)s in at least one replicate of fetal nucleic acid, said paternal haplotype informative SNPs are not present in the maternal genotype, thereby determining unique paternal SNPs identified in the fetus.

According another embodiment, said analyzing said replicate of fetal nucleic acid sequence comprises determining maternal haplotype informative SNPs in one or more replicates of fetal nucleic acid, thereby determining maternal haplotype in said fetus.

One skilled in the art would appreciate that in instances where parental homozygosity overlaps with a consensus haplotype, larger genetic regions may be analyzed, so as to increase the probability of heterozygote locus identification. In some embodiments, larger genetic regions include up to hundreds or thousands additional SNPs.

According to some embodiments, said method is for predicting an increased risk of a monogenic disease or disorder in a fetus of a pregnant female. According another embodiment, said maternal haplotype comprises a founder haplotype encompassing a founder mutation, said method being useful for predicting an increased risk of said founder mutation in said fetus. According another embodiment, said monogenic disease or disorder is caused by, or strongly associated with, a founder mutation. According another embodiment, said monogenic disease or disorder presents with autosomal recessive inheritance.

None limiting examples of diseases or disorders caused by, or strongly associated with, a founder mutation include Gaucher disease, cystic fibrosis, beta-thalassemia, sickle cell anemia, Amegakaryocytic Thrombocytopenia, Alpha 1-antitrypsin deficiency, Ataxia Telangiectasia, Autoimmune Polyglandular Syndrome, Bardet Biedl syndrome, Bloom syndrome, Canavan disease, Costeff syndrome, Cystinosis, Dihydrolipoamide dehydrogenase deficiency, Ellis-van Creveld syndrome, Familial Dysautonomia, Familial hyperinsulinemia, Fanconi anemia C, Glycogen Storage Disease Type Ia, Hermansky-Pudlak syndrome, Homocystinuria, autosomal recessive Hydrocephalus, Joubert syndrome 2, Leber congenital amaurosis, Leigh syndrome, Microcephaly with complex motor and sensory axonal neuropathy, Maple Syrup Urine Disease (MSUD), Megalencephalic leukoencephalopathy with subcortical cysts, Mitochondrial neurogastrointestinal encephalopathy syndrome, Mucolipidosis IV, Nemaline myopathy. Niemann-Pick disease A, Osteopetrosis, Pendred syndrome, Pontocerebellar hypoplasia type 1, Progressive cerebello-cerebral atrophy, Retinitis pigmentosa, Rothmund-Thomson syndrome, Senior-Loken syndrome, Tay-Sachs disease, Tyrosinemia, Usher syndrome I, Usher syndrome III, Walker Warburg syndrome and Zelweger syndrome. According to another embodiment, the present invention provides a kit for identifying and/or analyzing fetal haplotype with a high degree of confidence. In one embodiment, the kit comprises one or more components for sequencing a nucleic acid sample (e.g., fetal nucleic acid sequence) at a depth of at least 100× coverage.

The kits may include, in some embodiments, ligands and buffers for practicing the disclosed methods. The kits may include, in some embodiments, at least one vial, test tube, flask, bottle, syringe or the like.

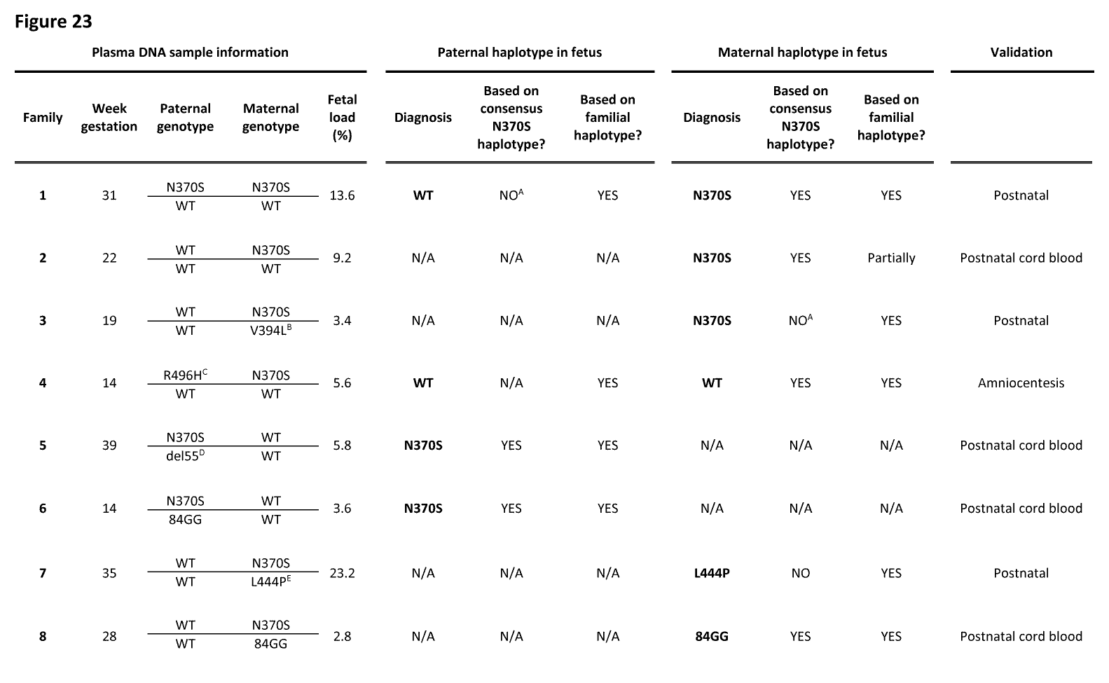

In another embodiment, there is provided a method for prenatal diagnosis of Gaucher type I. In another embodiment, said method comprises the method comprising: obtaining a fetal nucleic acid sequence sequenced, said fetal nucleic acid sequence being derived from plasma DNA samples obtained from a pregnant female; wherein at least one SNP listed in FIG. 23 indicates that said fetus is afflicted with Gaucher type I. In one embodiment, said fetus is a carrier of the N370S founder mutation.

As used herein, the term “Single Nucleotide Polymorphism” or “SNP” refers to a single nucleotide that may differ between the genomes of two members of the same species. The usage of the term should not imply any limit on the frequency with which each variant occurs.

The process of determining which specific nucleotide (i.e., allele) is present at each of one or more SNP positions is referred to as SNP genotyping. The present invention provides methods of SNP genotyping, such as for use in screening for a variety of disorders, or determining predisposition thereto, or determining responsiveness to a form of treatment, or prognosis, or in genome mapping or SNP association analysis.

According to one aspect the present invention provides a method for non-invasively predicting an increased risk of maternal and/or paternal haplotypes inherited by a fetus of a pregnant female, the method comprising: obtaining a fetal SNP genotype derived from DNA samples obtained from the pregnant female; and analyzing fetal SNP genotype, wherein at least 95% identity of said fetal SNP haplotype to a consensus haplotype indicates that said fetus is a carrier of a maternal and/or paternal haplotype; thereby predicting an increased risk of a maternal and/or paternal haplotype inherited by said fetus.

In another embodiment, determining at least part of a fetal genome could be used for paternity testing by comparing the deduced fetal genotype or haplotype with the genotype or haplotype of the alleged father.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues MoI. Biol. 2003 April; 5(2):43-60; Shi, “Technologies for individual genotyping: detection of genetic polymorphisms in drug targets and disease genes”, Am J Pharmacogenomics. 2002; 2(3): 197-205; and Kwok, “Methods for genotyping single nucleotide polymorphisms”, Annu Rev Genomics Hum Genet 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21.

Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (see, e.g., U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

In another embodiment, a “sequence” refers to a DNA sequence or a genetic sequence. It may refer to the primary, physical structure of the DNA molecule or strand in an individual. It may refer to the sequence of nucleotides found in that DNA molecule, or the complementary strand to the DNA molecule. It may refer to the information contained in the DNA molecule as its representation in silico.

In another embodiment, a “locus” refers to a particular region of interest on the DNA of an individual, which may refer to a SNP, the site of a possible insertion or deletion, or the site of some other relevant genetic variation. Disease-linked SNPs may also refer to disease-linked loci. Polymorphic Allele, also “Polymorphic Locus,” refers to an allele or locus where the genotype varies between individuals within a given species. Some examples of polymorphic alleles include single nucleotide polymorphisms, short tandem repeats, deletions, duplications, and inversions. Polymorphic Site refers to the specific nucleotides found in a polymorphic region that vary between individuals.

Haplotype refers to a combination of alleles at multiple loci that are typically inherited together on the same chromosome. Haplotype may refer to as few as two loci or to an entire chromosome depending on the number of recombination events that have occurred between a given set of loci. Haplotype can also refer to a set of SNPs on a single chromatid that are statistically associated.

Genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

“Genetic material” or “Genetic sample” refers to physical matter, such as tissue or blood, from one or more individuals comprising DNA or RNA.

Allelic data refers to a set of genotypic data concerning a set of one or more alleles. It may refer to the phased, haplotypic data. It may refer to SNP identities, and it may refer to the sequence data of the DNA, including insertions, deletions, repeats and mutations. It may include the parental origin of each allele.

Confidence refers to the statistical likelihood that the called SNP, allele or set of alleles correctly represents the real genetic state of the individual.

Homozygous refers to having similar alleles as corresponding chromosomal loci. Heterozygous refers to having dissimilar alleles as corresponding chromosomal loci.

Maternal Plasma refers to the plasma portion of the blood from a female who is pregnant. Parental context refers to the genetic state of a given SNP, on each of the two relevant chromosomes for one or both of the two parents of the target.

Clinical decision refers to any decision to take or not take an action that has an outcome that affects the health or survival of an individual. In the context of prenatal diagnosis, a clinical decision may refer to a decision to abort or not abort a fetus. A clinical decision may also refer to a decision to conduct further testing, to take actions to mitigate an undesirable phenotype, or to take actions to prepare for the birth of a child with abnormalities.

The term “HLA-type” refers to the complement of HLA antigens present on the cells of an individual. An individual's HLA-type may be used to predict favorable donor-recipient pairs for tissue transplant or blood transfusion or may be used as an indicator of the individual's susceptibility to certain diseases or conditions. In particular, an individual's HLA serotype can be used to predict compatibility between a blood transfusion donor and recipient. An HLA-type can be determined according to the proteins expressed from particular alleles of genes in the MEW region; for example an HLA-type can refer to specific HLA class I proteins or HLA class II proteins. Typically, genes that may be represented in an HLA-type include one or more genes selected from the group consisting of HLA-A, HLA-B, HLA-Cw, HLA-DR, HLA-DQ and HLA-DP. Terminology for specific HLA-types is usually expressed in accordance with reports released by the World Health Organization Committee on Nomenclature.

The term “HLA gene” as used herein, refers to a genomic nucleotide sequence that expresses an HLA class I or HLA class II proteins. Class I HLA genes include HLA-A, HLA-B and HLA-C, and class II HLA genes include HLA-DR, HLA-DQ, HLA-DQB1, and HLA-DP. The genes include a coding region which is a portion of the genomic sequence that is transcribed into mRNA and translated into a protein product. The genes further include portions of the genomic sequence that regulate expression of particular protein products. In another embodiment the present invention is a method for inferring fetal HLA genotype by comparison to a predetermined consensus haplotype.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Materials and Methods

Sample Collection and DNA Extraction

Pregnant Ashkenazi Jewish (AJ) couples, carrying mutation(s) in the GBA gene, were recruited at the Shaare Zedek Medical Center (SZMC) Gaucher Clinic. Peripheral blood samples were collected from each couple, relevant mutation carrier family members, 8 unrelated AJ GBA N370S homozygotes, and 3 unrelated AJ GBA N370S heterozygote duos. Genomic DNA was then prepared from all samples using the FlexiGene DNA kit (QIAGEN) according to the manufacturer's protocol. For pregnant female indices, plasma was separated from peripheral blood by centrifugation at 1,900×g for 10 minutes at 4° C. The plasma supernatant was then recentrifuged at 16,000×g for 10 minutes at 4° C. and 3 ml of the resulting supernatant was used for cell-free DNA extraction with the QIAamp Circulating Nucleic Acid kit (QIAGEN) according to the manufacturer's protocol. The maternal plasma DNA extracts were then pre-amplified, in duplicate, with the SurePlex Amplification System (Illumina) ahead of downstream processing. All familial mutations in GBA were Sanger sequence verified prior to commencement of the study. Ethical approval for the study, including usage of materials from human subjects, was obtained from the local institutional review board and written informed consent was obtained from all study participants.

### Next Generation Sequencing (NGS) of GBA-Flanking Single Nucleotide Polymorphisms (SNPs)

Two TruSeq Custom Amplicon panels were designed with Design Studio software (Illumina) to amplify and sequence GBA-flanking SNPs in all samples. The smaller panel sequenced 490 SNPs and the larger panel sequenced 5,000 SNPs. Indexed next generation sequencing libraries were prepared and normalized according to the manufacturer's protocol (Illumina) followed by 2×150 bp pair-end sequencing on a MiSeq (small panel) or NextSeq 500 (large panel) instrument (Illumina) to a mean depth of at least 500× or 3800× for genomic and plasma DNA samples, respectively. After sequencing runs, the data were aligned to target sequences on the human reference genome (hg19) using MiSeq Reporter software (Illumina) for the small panel or the TruSeq Amplicon v1.1 app on BaseSpace (https://basespace.illumina.com/) for the large panel. Genotyping data was extracted from each alignment using the SAMtools mpileup program to yield sample-specific SNP genotype profiles and then the SNPs were annotated by snpEff with dbSNP138 (small panel) or dbSNP141 (large panel). These profiles were then combined into single family-specific .csv files using in-house software so as to facilitate familial and fetal linkage analysis (see below). Prior to linkage analysis, non-GBA flanking SNP calls and SNP calls on heavily self-chained genomic segments were removed. Genomic DNA SNP genotype calls were categorized into one of 3 distinct classifications based on the percentage of non-reference genome allele (B allele) sequencing reads at each locus: homozygote reference allele (AA; 0%-20% B allele reads); homozygote non-reference allele (BB; 80%-100% B allele reads); or heterozygote (AB; 30%-70% B allele reads). Any loci that did not meet these classification criteria were excluded from further downstream analysis. As a rule, parental haplotypes were constructed with SNPs for which the parent was heterozygous and at least one of his/her first degree relatives was homozygous.

Construction of Consensus AJ N370S and Familial Haplotypes

The initial consensus AJ N370S GBA-flanking haplotype was constructed by performing homozygosity mapping with custom SNP small panel NGS datasets from 7 unrelated AJ N370S homozygotes (14 N370S chromosomes). Subsequently, 6 more AJ N370S haplotypes were derived from linkage analysis on SNP NGS datasets from 6 unrelated AJ N370S mutation carrier duos. Each linkage-based N370S haplotype was then crossed with the consensus sequence derived from homozygosity mapping to identify inconsistencies. These sequence discrepancies were then used to mark consensus AJ N370S founder haplotype cut-offs (based on 20 N370S chromosomes, altogether, after the completion of all data intersections). The larger consensus AJ N370S GBA-flanking haplotype was constructed by performing homozygosity mapping with custom SNP large panel NGS datasets from 8 unrelated AJ N370S homozygotes (16 N370S chromosomes). Subsequently, 12 more AJ N370S haplotypes were derived from linkage analysis on SNP NGS datasets from 12 unrelated AJ N370S mutation carrier duos. The final consensus AJ N370S founder haplotype cut-offs (based on 28 N370S chromosomes, altogether, after the completion of all data intersections) were then set as described above regarding the initial consensus haplotype construct. Identification of fetal alleles in maternal plasma DNA

In order to construct credible small fetal haplotypes (composed of <5 SNPs) with the small SNP sequencing panel, plasma DNA samples were sequenced in duplicate at high depth (>3,000×mean coverage) so as to augment statistical confidence in each individual fetal SNP genotype call. In all, four different combinations of parental SNP genotypes were analyzed in plasma DNA: A) Error rate informative (father and mother [of the fetus] both homozygote “AA”); B) Dosage informative (father and mother homozygote for opposite alleles); C) Paternal haplotype informative (father heterozygote and mother homozygote); and D) Maternal haplotype informative SNPs (mother heterozygote and father homozygote). Error rate informative SNPs measured the sequencing error rate in plasma DNA samples by assessing the appearance of biologically impossible SNP reads. At >1000×read depth, error rates of 0.6%+/−0.6% were measured in plasma DNA samples. Dosage informative SNPs (denoted heretofore as “SNP I”) measured the paternal portion of fetal plasma DNA by determining the fraction of paternal alleles per maternal alleles. These SNPs also confirmed the presence of fetal DNA in maternal plasma. Paternal haplotype informative SNPs (denoted heretofore as “SNP II”) feature a unique nucleotide in the fetus' father that is not present in the maternal genotype. When identified in maternal plasma DNA, the paternal unique allele is expected to comprise the same fraction as those of paternal alleles in dosage informative SNPs. In general, the paternal haplotype of the fetus was deduced wherever the father's unique SNP II allele was identified in one of 2 plasma DNA replicates (at a SNP position with >1000×sequencing depth) with relatively high frequency (>2σ from the mean sequencing error rate as determined from error rate informative SNPs) in maternal plasma DNA. The computed sensitivity/specificity scores for this method are provided as a function of the number of unique paternal SNPs identified in the fetus (see Table 1).

TABLE 1
Simulated sensitivity/specificity for unique paternal allele diagnosis

| No. SNPs in fetal haplotype | Sensitivity/SpecificityA |
|---|---|
| 1 | 94.97% |
| 2 | 99.72% |
| 3 | 99.96% |
| 4 | 99.97% |
| 5 | 100.00% |
| 6 | 100.00% |
| 7 | 100.00% |
| 8 | 100.00% |
| 9 | 100.00% |
| 10 | 100.00% |

AThe formula for these calculations was as follows: [1 − ([(0.5)(er)] + [(0.5)(er)])n] where “n” represents the number of SNPs in the fetal haplotype and “er” represents the chance (which is 5%) of unique paternal allele detection at 2σ from the sequencing error rate as determined from error rate informative SNP sequences, as described herein above. For 1 to 4 SNP haplotypes, a 0.03% correction was applied to account for the sex-specific male recombination rate in the +/−250 kb genomic region surrounding GBA, but if longer haplotypes do not flank the mutation, this correction should continue to be applied.

For plasma DNA samples with high fetal dosage (>30% paternal fetal fraction), the paternal haplotype in the fetus was also deduced from non-unique SNP II alleles (with >500× coverage) for which there were no discrepancies between replicate fetal haplotype calls. The computed sensitivity/specificity scores for this method are provided as a function of the number of non-unique paternal SNPs identified in the fetus (see Table 2).

TABLE 2
Simulated sensitivity/specificity for non-unique paternal allele diagnosis

| No. SNPs in fetal haplotype | Sensitivity/Specificity^{A} |
|---|---|
| 1 | 77.41% |
| 2 | 94.88% |
| 3 | 98.82% |
| 4 | 99.71% |
| 5 | 99.94% |
| 6 | 99.99% |
| 7 | 100.00% |
| 8 | 100.00% |
| 9 | 100.00% |
| 10 | 100.00% |

^{A}The formula for these calculations was as follows: [1 − ([(0.5)(1 − er)]²)ⁿ] where “n” represents the number of SNPs in the fetal haplotype and “er” represents the chance (which is 5%) of unique paternal allele detection at 2σ from the sequencing error rate as determined from error rate informative SNP sequences. For 1 to 4 SNP haplotypes, a 0.03% correction was applied to account for the sex-specific male recombination rate in the +/−250 kb genomic region surrounding GBA, but if longer haplotypes do not flank the mutation, this correction should continue to be applied.

Maternal haplotype informative SNPs (denoted heretofore as “SNP III”) were used to determine the maternal haplotype in the fetus at >1000× sequencing coverage. These SNPs indicated a heterozygous fetal genotype when allele-allele ratios were balanced, and a homozygous fetal genotype when these ratios were imbalanced by a number >3σ from the mean sequencing error rate (as determined from error rate informative SNPs). Depending on the father's homozygous allele, the maternal fetal allele was deduced based on the presence or absence of skewing (<50% non-reference nucleotide skewed representation if the father was homozygote A [for the reference nucleotide]; >50% non-reference nucleotide skewed if the father was homozygote B [for the non-reference nucleotide]) in maternal heterozygous SNP III loci on both plasma DNA replicates. The computed sensitivity/specificity scores for this method are provided as a function of the number of maternal haplotyped SNPs identified in the fetus (see Table 3).

TABLE 3
Simulated sensitivity/specificity for maternal allele diagnosis

| No. SNPs in fetal | |
|---|---|
| haplotype | Sensitivity/Specificity^{A} |
| 1 | 74.93% |
| 2 | 93.68% |
| 3 | 98.37% |
| 4 | 99.54% |
| 5 | 99.90% |
| 6 | 99.98% |
| 7 | 99.99% |
| 8 | 100.00% |
| 9 | 100.00% |
| 10 | 100.00% |

^{A}The formula for these calculations was as follows: [1 − [(0.5)²]ⁿ] where “n” represents the number of SNPs in the fetal haplotype. For 1 to 4 SNP haplotypes, a 0.07% correction was applied to account for the sex-specific female recombination rate in the +/−250 kb GBA region but if longer haplotypes do not flank the mutation, this correction should continue to be applied.

All parental SNP combinations that did not fall within the above guidelines were not utilized in this study. In order to construct large fetal haplotypes (composed of >5 SNPs) with the large SNP sequencing panel, plasma DNA samples were analyzed as above with the following modifications. Error rate informative SNPs indicated a 1% error rate at read depths exceeding 100×. Accordingly, paternal haplotype informative and maternal haplotype informative SNPs were assessed from a minimum read depth of 100 whereupon only skewing exceeding 1% B-allele frequency in plasma DNA with respect to maternal DNA (at a particular locus) was considered significant enough for incorporation into the fetal haplotype. This filter was applied so as to reduce genotyping errors emerging from either sequencing error and/or off-target sequence contamination.

Ultimately, fetal diagnosis was achieved after comparing the paternal and maternal cell-free fetal DNA (cffDNA) haplotypes with family-based and/or N370S consensus or near consensus haplotypes as relevant. Altogether, the entire noninvasive NGS-based prenatal test, from blood sample processing to fetal diagnosis, was completed in 5 work days. In addition, all diagnoses were confirmed by post-natal genetic testing. For family 1, allelic inheritance of the N370S mutation was further confirmed by postnatal linkage analysis with short tandem repeat (STR) markers.

### Example 1: Noninvasive Prenatal Diagnosis of an Autosomal Recessive Founder Mutation

Study Description

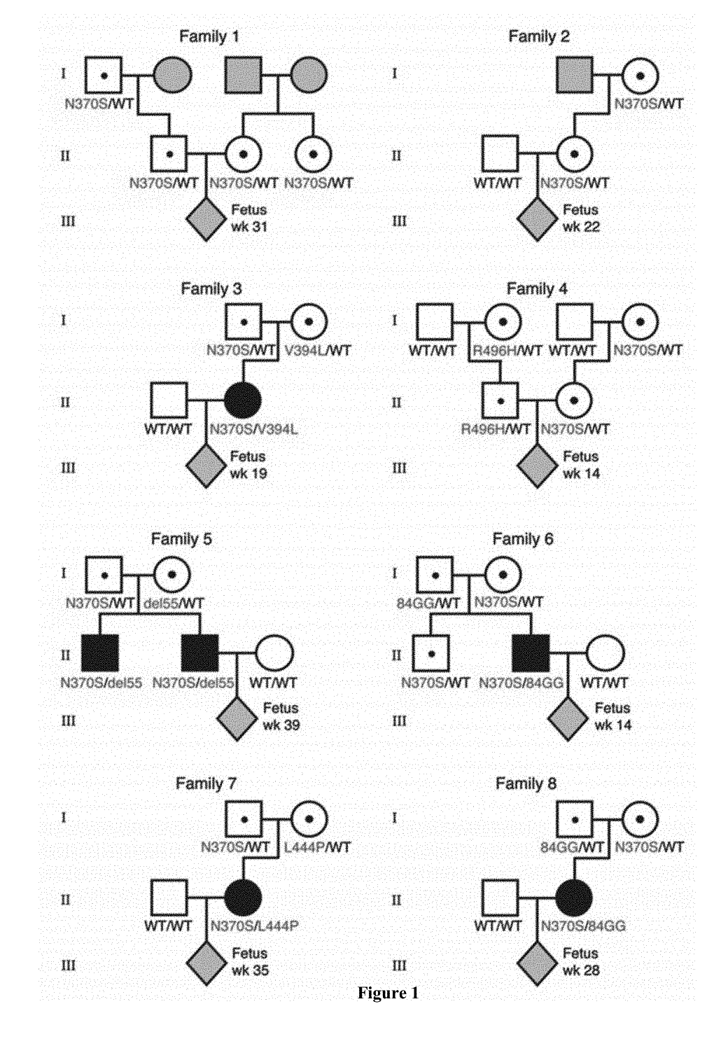

Eight pregnant AJ couples, of which one or both partners were heteroallelic carriers of GBA N370S, were enrolled in the study (FIG. 1). Although families 1 and 4 were at risk of giving birth to a homozygote N370S child (unlike families 2, 3, 5, 6, 7, and 8 in which one parent of the fetus did not carry any mutation in GBA), all couples were tested strictly for proof-of-principle purposes. Plasma samples were collected from female participants at the time points indicated in FIG. 1 for DNA extraction and targeted high-throughput sequencing of GBA-flanking SNPs. To enhance diagnostic accuracy, the inventors elected to defer direct mutation sequencing in favor of a more specific and sensitive linkage-based analytical regimen. This methodology strengthens diagnostic confidence with increasing fetal haplotype size (measured by the number of SNPs in the inferred fetal haplotype) (Tables 1-3). To accomplish this goal, the inventors first sequenced GBA-flanking SNPs (up to ±250 kb distance from GBA) of the parents and their first-degree relatives in families 1 and 2, so as to construct parental haplotypes. However, these family-based haplotypes were of limited size (Table 4). Therefore, a larger haplotype sequence was sought to aid fetal diagnosis by mapping a consensus N370S founder region surrounding the GBA gene.

TABLE 4
Parental family-based haplotype information

| | Paternal familial haplotype data | | | | Maternal familial haplotype data | | | |
|---|---|---|---|---|---|---|---|---|
| | | Paternal | | | | Maternal | | |
| | | family | Genotype | No. of | | family | Genotype | No. of |
| | | member | of paternal | SNPs in | | member | of maternal | SNPs in |
| | Paternal | used for | family | linked | Maternal | used for | family | linked |
| Family | genotype | linkage | member | haplotype | genotype | linkage | member | haplotype |
| 1 | N370S/WT | father | N370S/WT | 3 | N370S/WT | sister | N370S/WT | 43 |
| 2 | WT/WT | N/A | N/A | N/A | N370S/WT | mother | N370S/WT | 11 |

Fine Mapping of the Consensus AJ N370S Founder Haplotype Region.

To fine map the N370S founder region, the inventors sequenced 7 unrelated homoallelic AJ mutation carriers on the targeted GBA-flanking SNP panel. Six of these homoallelic patients with type I Gaucher disease were homozygotic for all 490 SNPs on the initial sequencing panel. The seventh sample shared the same haplotype within and 3′ to GBA, but a heterozygous region was clearly identified

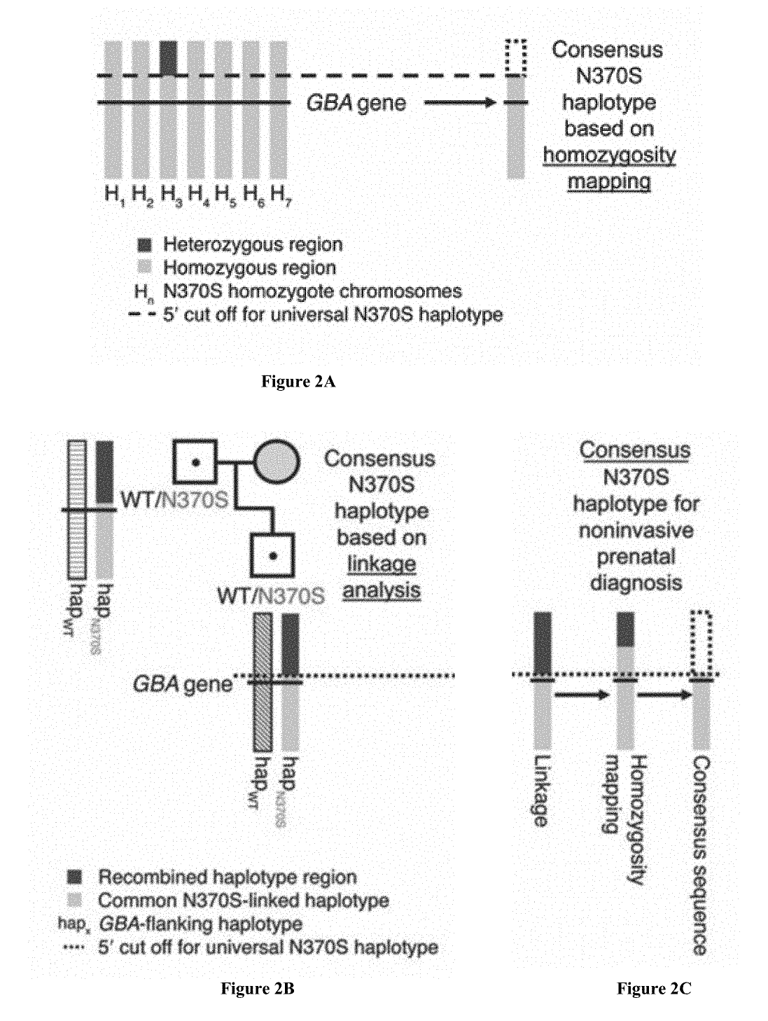

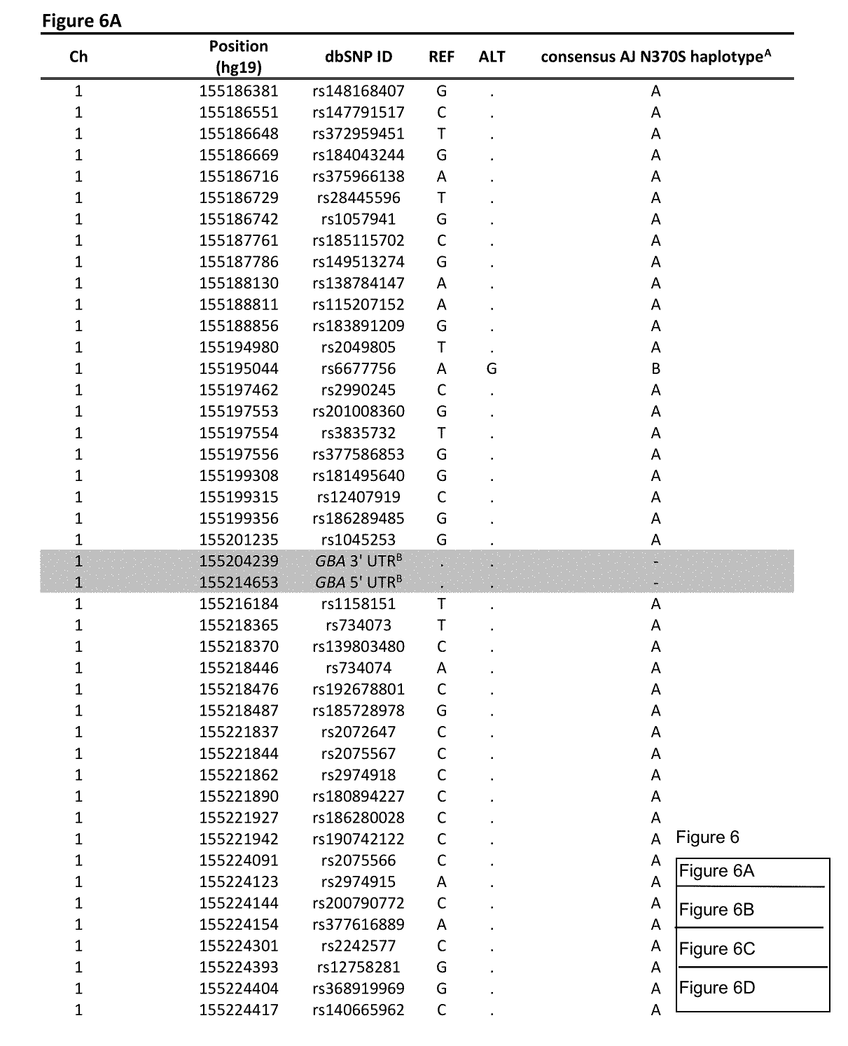

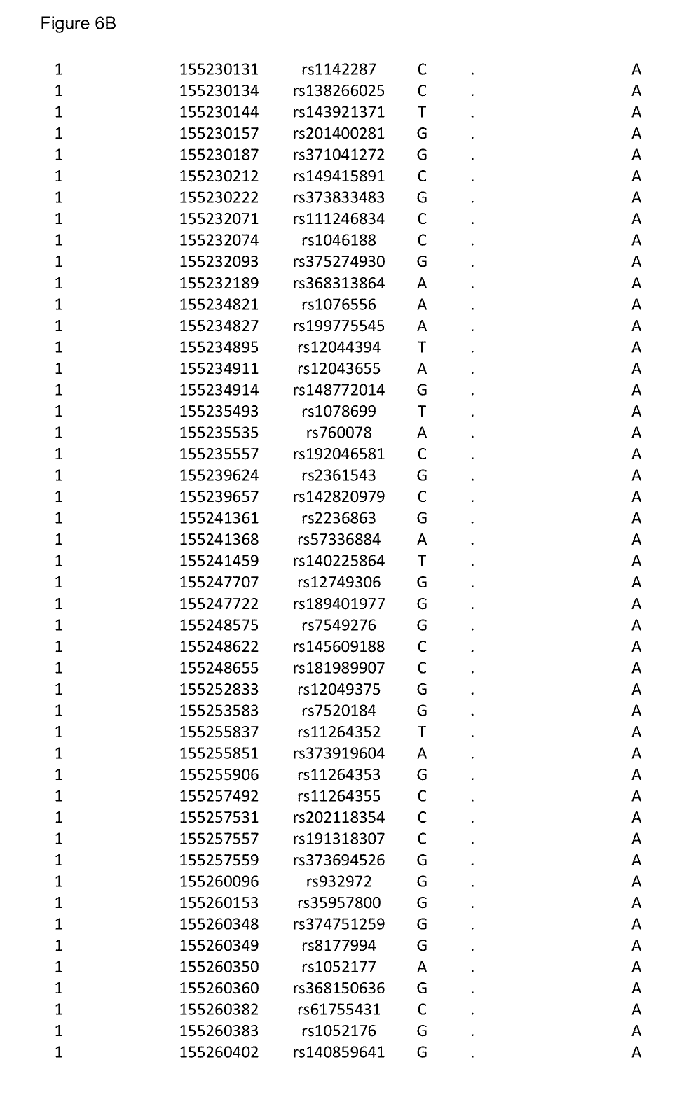

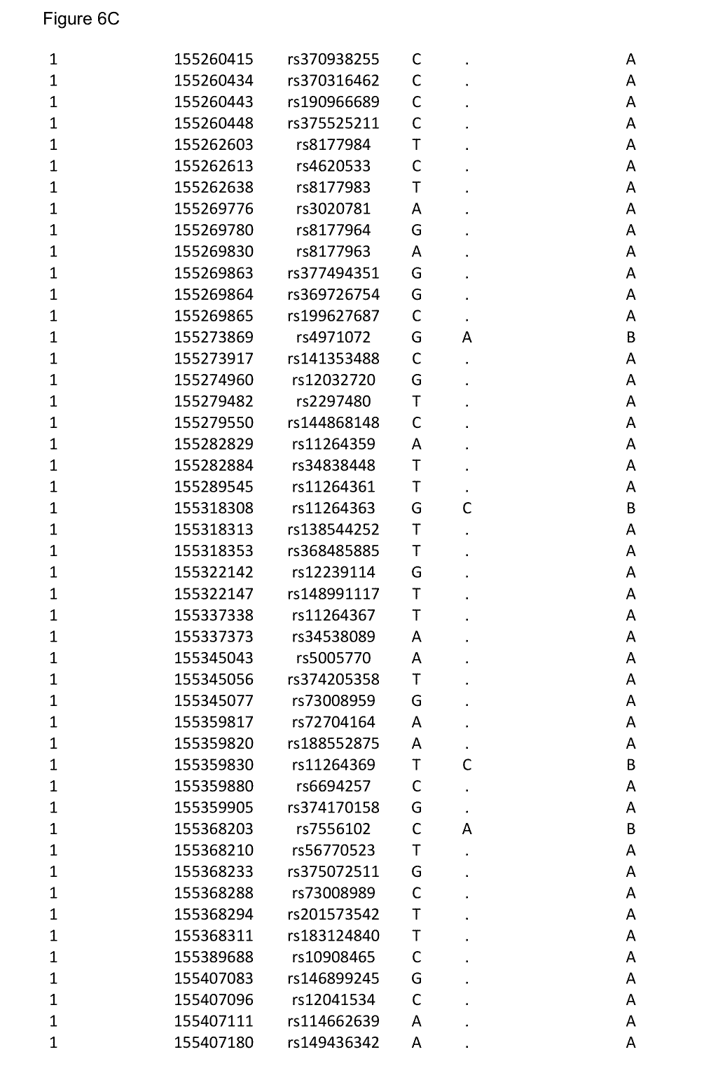

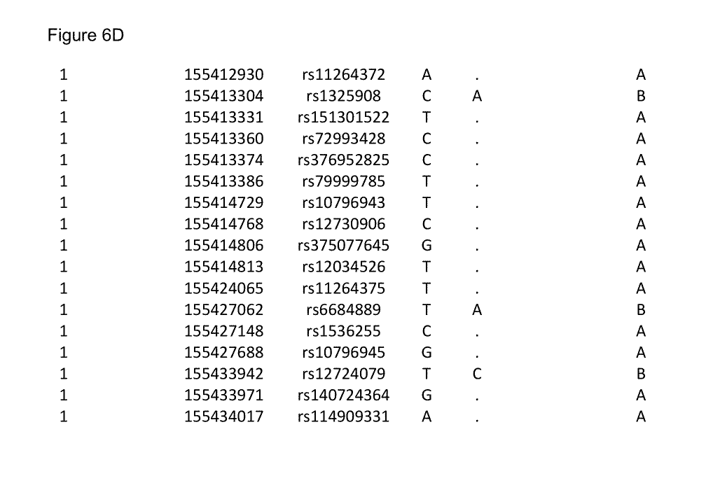

144,388 nucleotides 5′ to the gene and beyond (at rs2306124, dbSNP 138). Hence, this sample was used to demarcate a preliminary consensus founder haplotype (FIG. 2A). To further clarify the N370S sequence, the inventors then crossed the preliminary version with linkage-based N370S haplotypes from the families under investigation in this study in addition to 3 other unrelated heteroallelic AJ N370S mutation carrier duos (2 first-degree relatives, each of whom carries the same mutation). This analysis identified a recombined region only 17,858 nucleotides upstream of GBA (at rs148168407, dbSNP 138) (FIG. 2B), but remarkably, not a single recombination event was identified in the entire 219-kb region downstream of GBA in any of the 20 N370S chromosomes analyzed. Not coincidentally, this 3′ conserved region has been previously characterized as a non-recombination hot spot by the HapMap Consortium. Moreover, previous studies have identified a conserved AJ N370S founder haplotype that extends even further downstream of GBA. Nevertheless, although it is likely that the founder haplotype is longer than that initially mapped, the SNP-sequencing panel still successfully linked a sizable amount of GBA-flanking SNPs (153 altogether) to a consensus AJ haplotype sequence (FIG. 2C and FIGS. 6A-D). The next question was to determine whether this population-based haplotype could be used as a diagnostic tool for NIPD.

Preliminary NIPD of an Autosomal Recessive Founder Mutation.

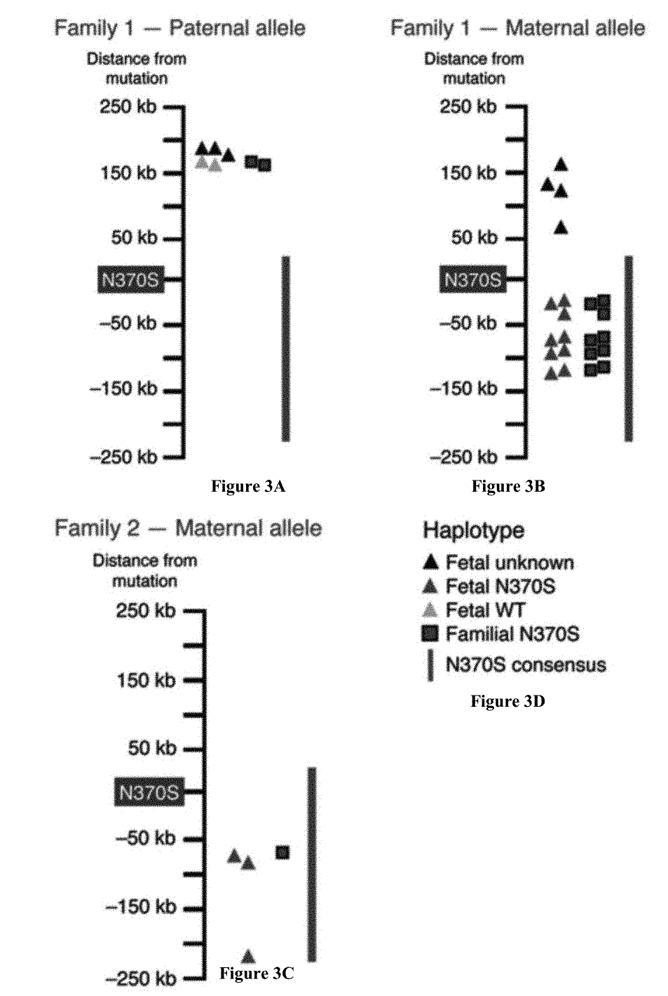

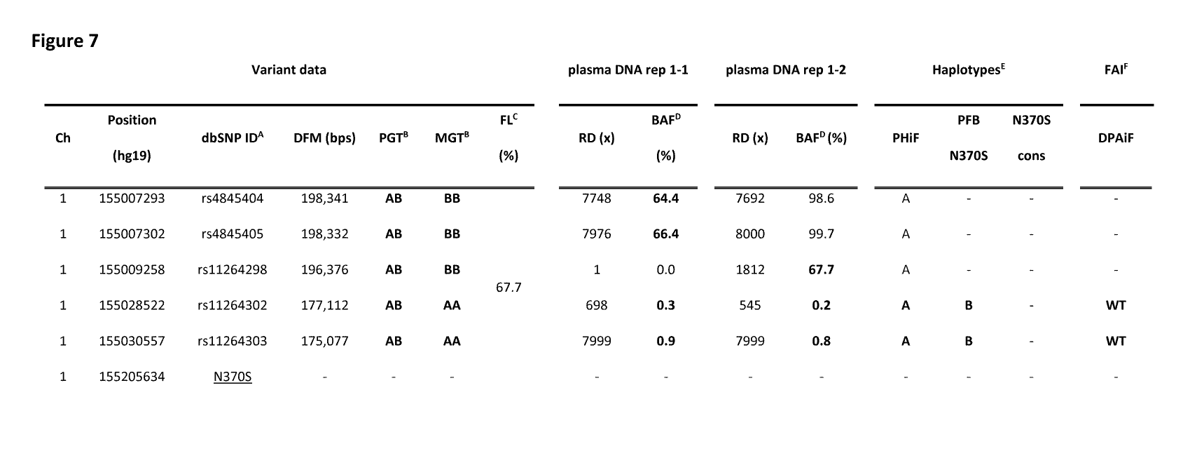

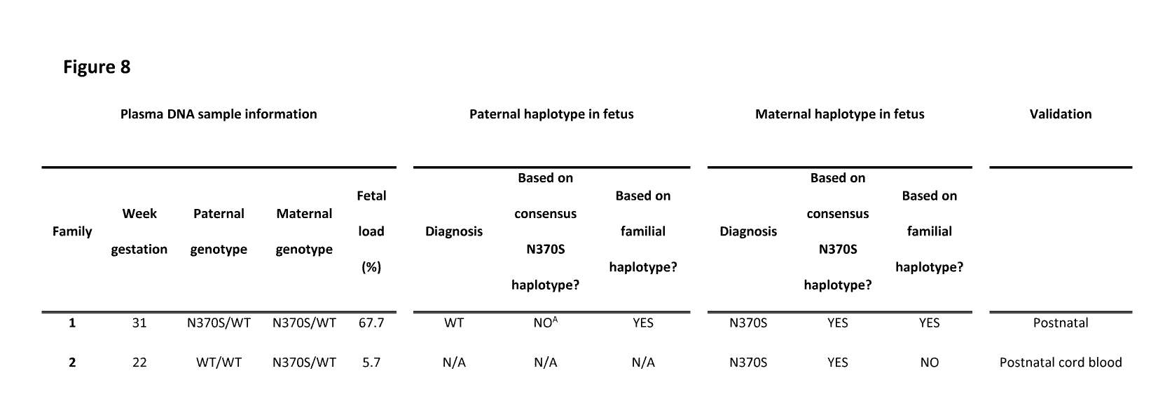

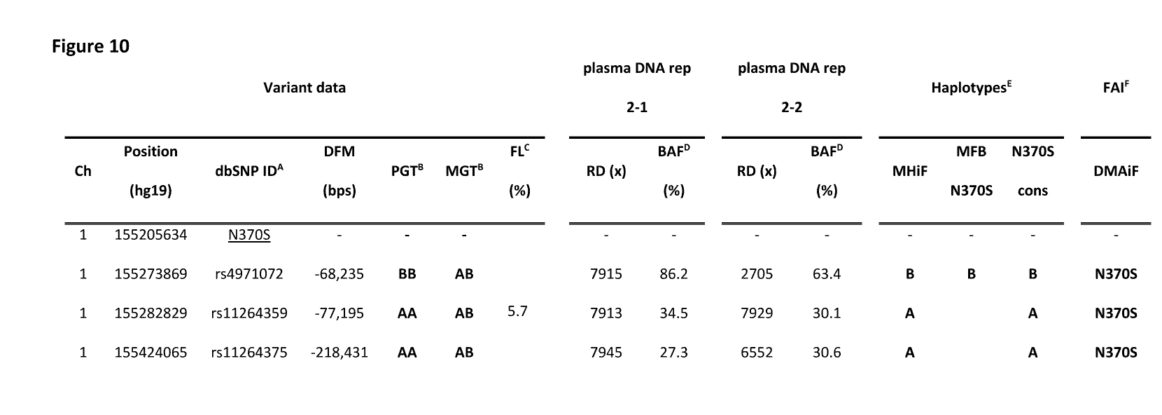

For pilot testing, families 1 and 2 offered 3 different avenues with which to assess the utility of the consensus N370S haplotype. This was because both parents (of the fetus) in family 1 were N370S carriers in addition to the mother in family 2 (FIG. 1). For family 1, the N370S carrier father was completely homozygous for the entire consensus N370S sequence. This precluded the use of the N370S haplotype for NIPD of his allele. Nonetheless, the familial mutation-linked haplotype of the father in family 1 did facilitate the identification of his WT allele in the fetus (FIG. 3A and FIGS. 7 and 8). Regarding the maternal alleles in families 1 and 2, the consensus N370S haplotype proved to be quite valuable. The family-based maternal N370S-linked haplotype was clearly identified in family 1 plasma DNA. This fetal haplotype was completely concordant with the consensus N370S haplotype (FIG. 3B and FIGS. 8 and 9). For family 2, the family-based maternal N370S haplotype could not reliably discern which allele was transmitted to the fetus because the fetal haplotype was determined on differing SNP positions. On the other hand, the longer consensus N370S haplotype clearly matched the inferred maternal haplotype in the fetus, indicating inheritance of the N370S allele (FIG. 3C and FIGS. 8 and 10). Thus, in this case, the consensus N370S sequence was crucial to the diagnosis of the maternal allele in the family 2 fetus.

Extended Fine Mapping of the Consensus AJ N370S Founder Haplotype Region.

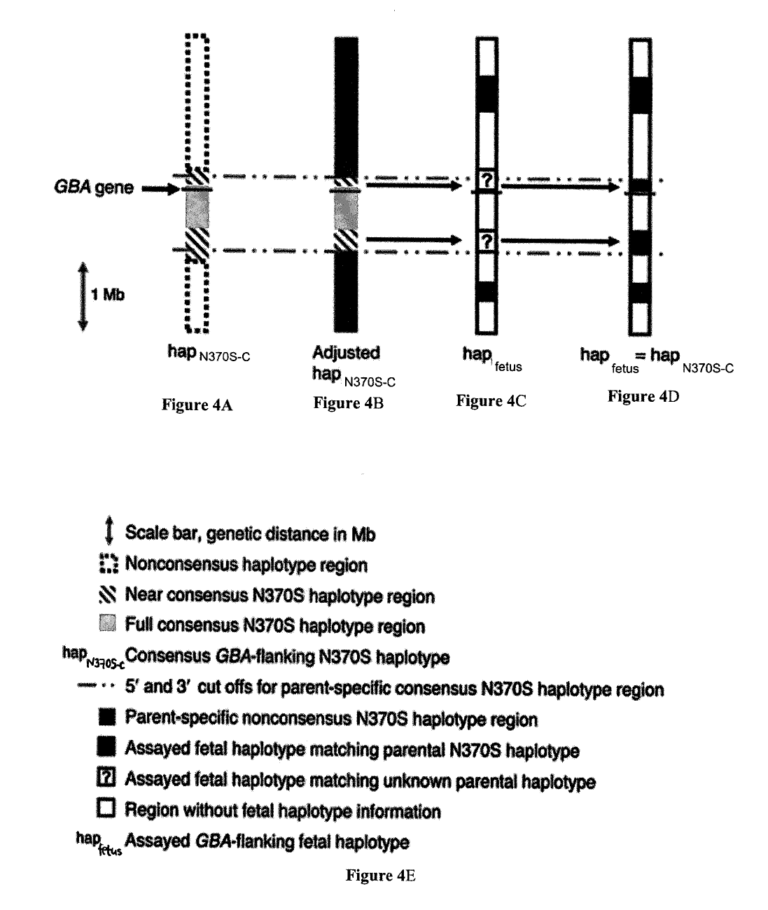

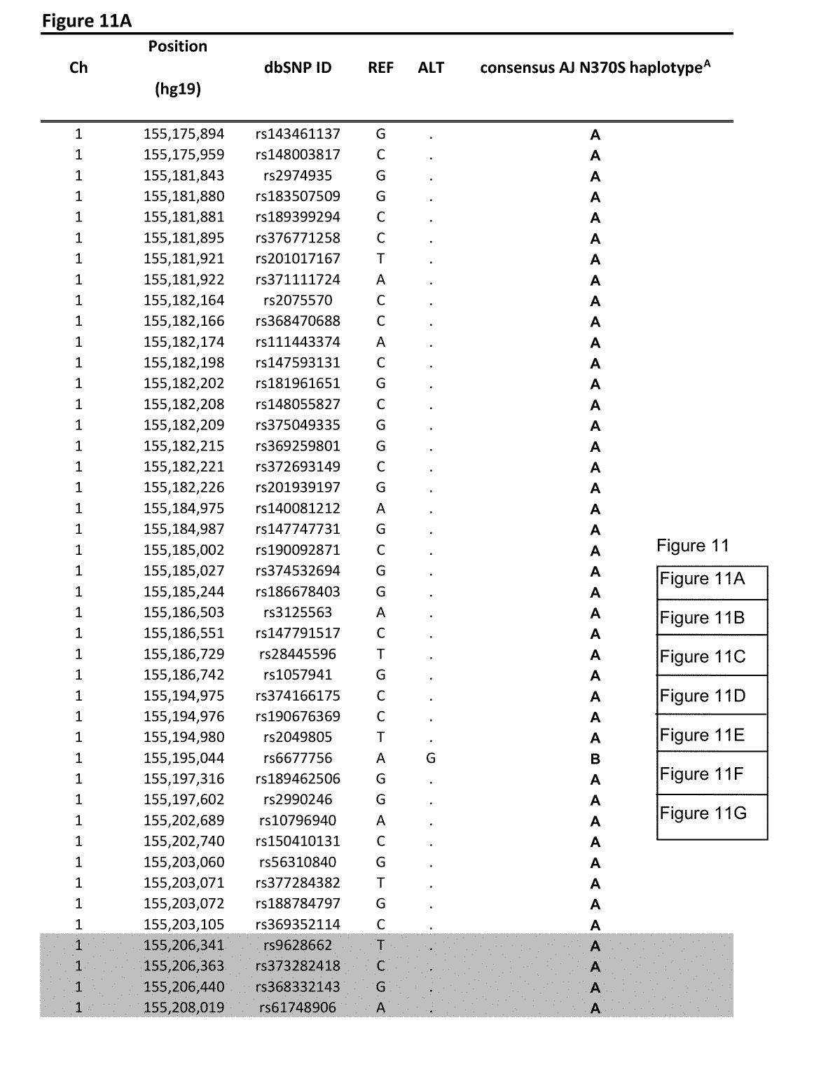

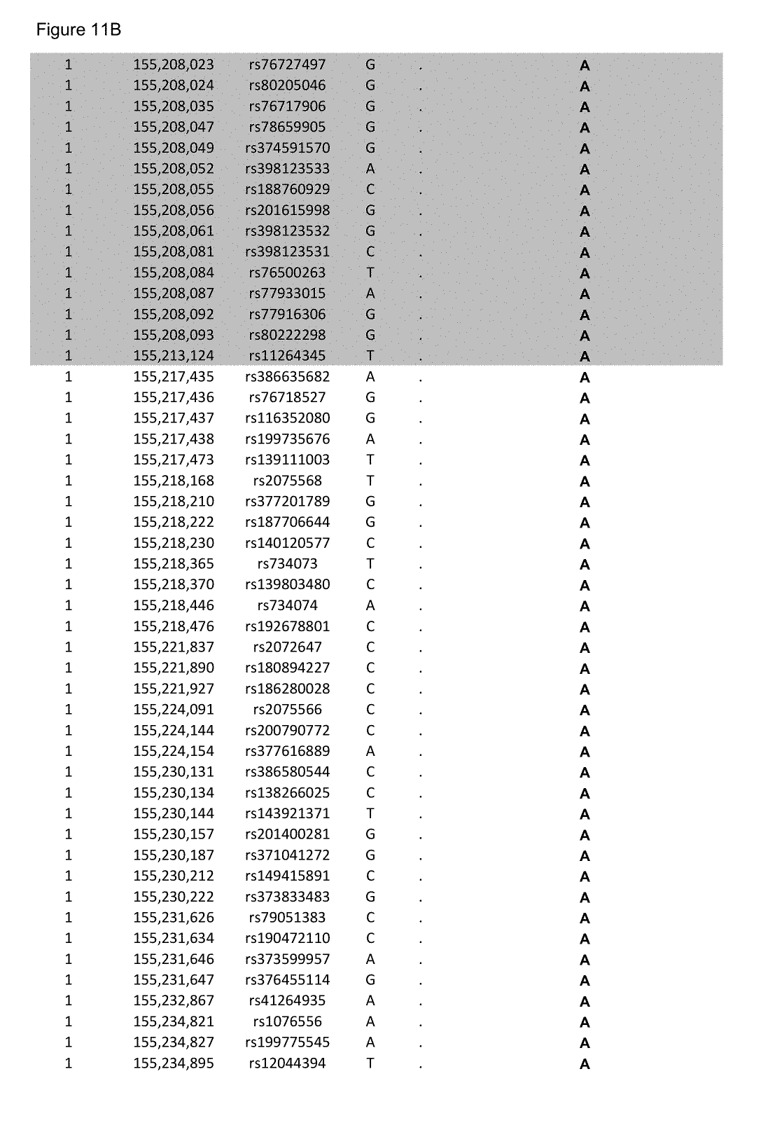

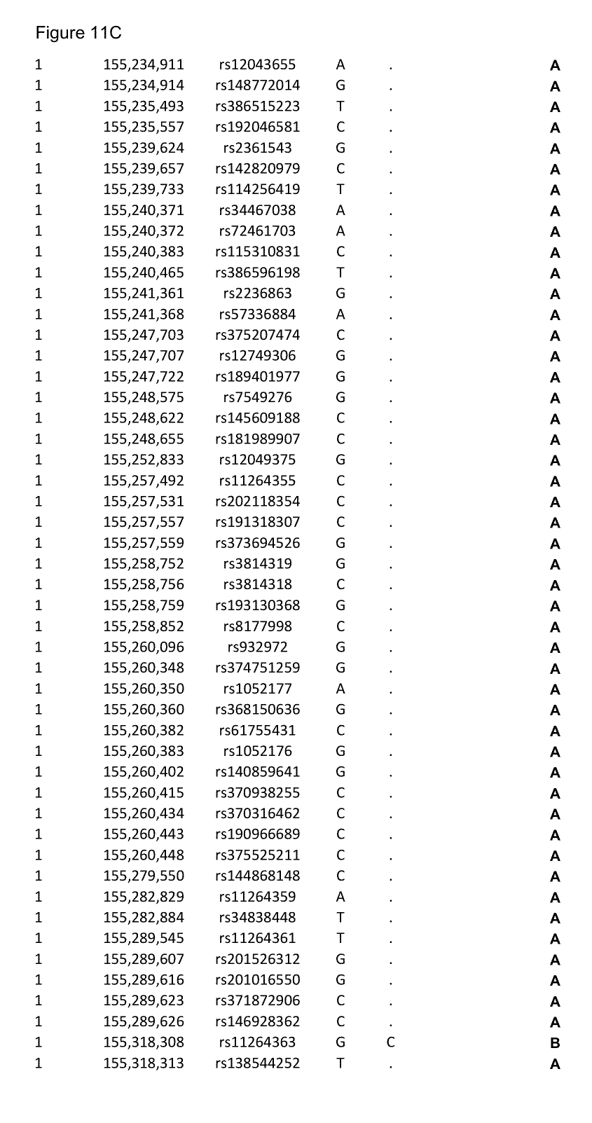

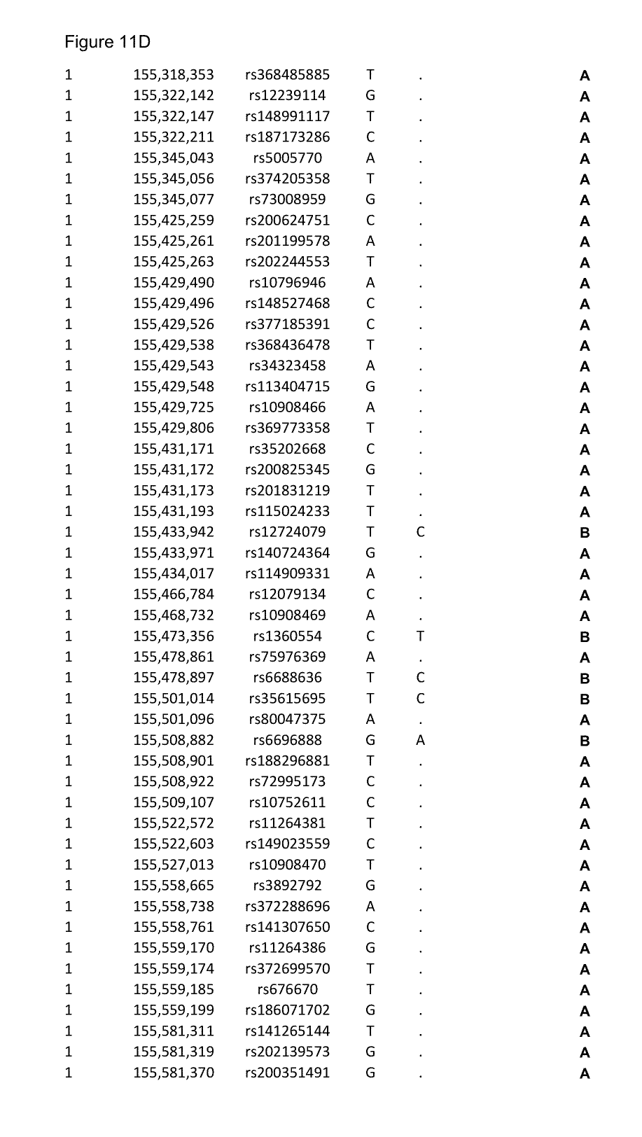

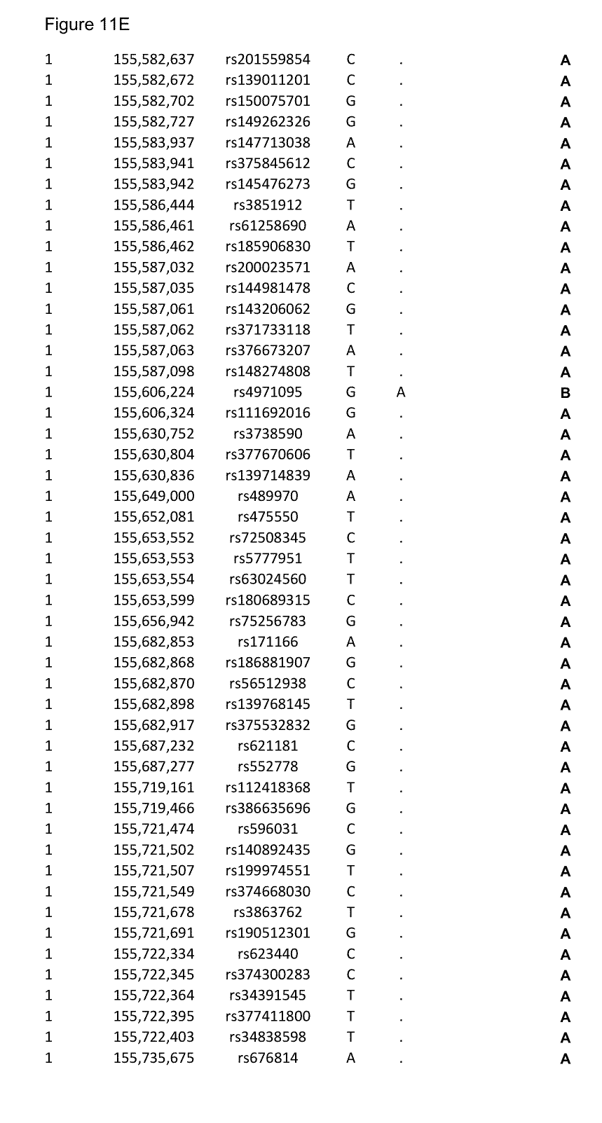

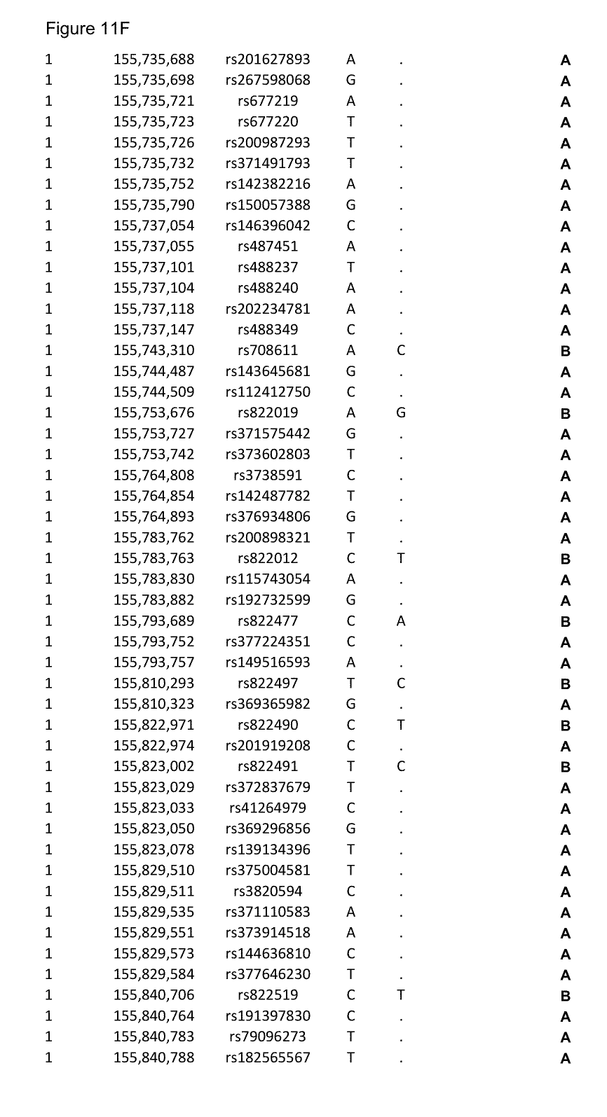

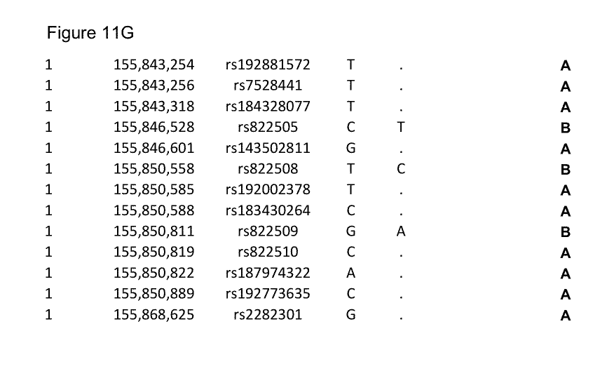

Although initial testing of families 1 and 2 showed promising results regarding the utility of the consensus N370S haplotype for incorporation into NIPD, it was clear that for expanded N370S testing in a clinical setting a more sophisticated sequencing panel would be required to facilitate setup of a universal assay for noninvasive prenatal Gaucher disease testing. The concerns with the initial 490-SNP sequencing panel were 4-fold. As evidenced by HapMap and deCode data, meiotic recombination is quite infrequent in the immediate human GBA-flanking locus (250 kb), which was the small target of the pilot sequencing panel. In this genomic context, homozygosity of an N370S mutation carrier parent, such as in the family 1 father, would be expected to occur commonly because DNA is rearranged at a reduced rate in the peri-GBA locus. Along these lines, low recombination rates translate into low genotypic complexity, which, in turn, leads to limited availability of linkage-informative SNPs, which are crucial to fetal haplotyping. Thus, small family-based haplotypes, which generally handicap fetal haplotyping, such as that of the family 1 father (3 SNPs) and that of the family 2 mother (11 SNPs; Table 4), would be predicted to represent the majority as opposed to the minority of cases. Another reason to consider looking beyond a distance of 250 kb from GBA would be to complete fine mapping of the 3′ boundary of the consensus N370S sequence, which proved so beneficial for fetal typing of family 1 and 2 maternal N370S-paired alleles. Finally, N370S aside, the implementation of a larger targeted sequencing panel should hypothetically be used to diagnose any mutation in GBA via familial linkage analysis, regardless of whether the mutation is a founder allele or not. For all these aforementioned reasons, a newer and much improved targeted deep-sequencing panel was designed to sequence 10 times the amount of GBA-flanking SNPs (˜5,000 SNPs) across an 8-fold-sized genomic region (GBA±2 Mb) before moving forward with NIPD for other families in the study. The first priority, in terms of test implementation, was to use the new expanded sequencing panel to complete fine mapping of the founder N370S haplotype. As mentioned above, the original sequencing panel successfully demarcated a 5′ boundary for the consensus sequence that was approximately 17 kb upstream of GBA and at least 219 kb downstream. When repeating the same exercise (as that described in FIGS. 2A-C) using the large sequencing panel, the 5′ boundary for the consensus haplotype mapped approximately 28 kb upstream of GBA (at SNP rs914615, dbSNP141). This 11 kb discrepancy between fine-map boundaries is quite remarkable, given that, due to technical reasons, the newer panel did not incorporate many of the SNPs sequenced previously with the older panel. Nevertheless, the preliminary 5′ cutoff, based only on N370S homozygotes, strikingly mapped to the exact same SNP position (SNP r52306124) 144,388 nucleotides 5′ to GBA in both panels. Thus, given this concordance between old and new sequencing panels regarding the 5′ N370S consensus sequence, it was especially edifying that the 3′ boundary of the haplotype fine mapped to a position that is roughly 650 kb downstream of GBA (at SNP rs1055184, dbSNP 141) by the new and improved panel. This expanded N370S-linked 670-kb-sized sequence (composed of 301 SNPs; FIGS. 11A-G) would already seem quite large for what is considered to be an ancient founder allele. Yet, remarkably, previous studies have shown that the N370S haplotype should, in fact, extend much further downstream from GBA, up to a full Mb from the gene. Indeed, after careful scrutiny of the new sequencing data, the inventors found that, among 16 sequenced N370S chromosomes from 8 N370S homozygotes, 15 chromosomes shared a near-consensus haplotype that extended 1.1 Mb 3′ to GBA (FIG. 4 A). Therefore, it was postulated that, if a 250-kb consensus sequence could be used to haplotype fetal alleles in families 1 and 2 (as in FIG. 3A-D), then a 1.1-Mb sequence might prove even more useful for typing of N370S chromosomes in most mutation carrier families in general.

To make effective use of the expanded near consensus N370S haplotype without allowing haplotype errors to corrupt downstream fetal analysis, the inventors carefully inspected each N370S chromosome in all mutation carrier parents in the study (families 1 through 8) using the large sequencing panel. It was found that, in some cases, recombination was detected in the true parent specific N370S-linked sequence with respect to the founder mutation near consensus haplotype (FIG. 4B). These discrepancies were applied on an allele-specific basis toward refinement of the consensus sequence, so that it could be used for the analysis of fetal haplotypes, which were not phased by conventional family based linkage analysis (FIG. 4C). In such scenarios, the parent-specific near-consensus N370S haplotype was appropriated for the resolution of unphased fetal haplotypes to increase confidence in the final NIPD test result (FIG. 4D). For example, if an N370S carrier mother and her immediate family members (whose samples were used for standard linkage analysis) were all heterozygous for the same SNP loci, the mother's genotypes were considered informative, even though linkage could not set phase on her N370S-linked haplotype. In this case, it was possible that the mother's unphased SNPs were located within the fine-mapped mother-specific consensus N370S haplotype (as in FIG. 4B) and genotyped in her fetus (as in FIG. 4C). When this occurs, the correct haplotype can be identified in the fetus, even though conventional family-based linkage analysis fails. More examples of this new approach to NIPD will be illustrated below.

NIPD of GBA N370S Using an Improved Targeted Sequencing Panel.

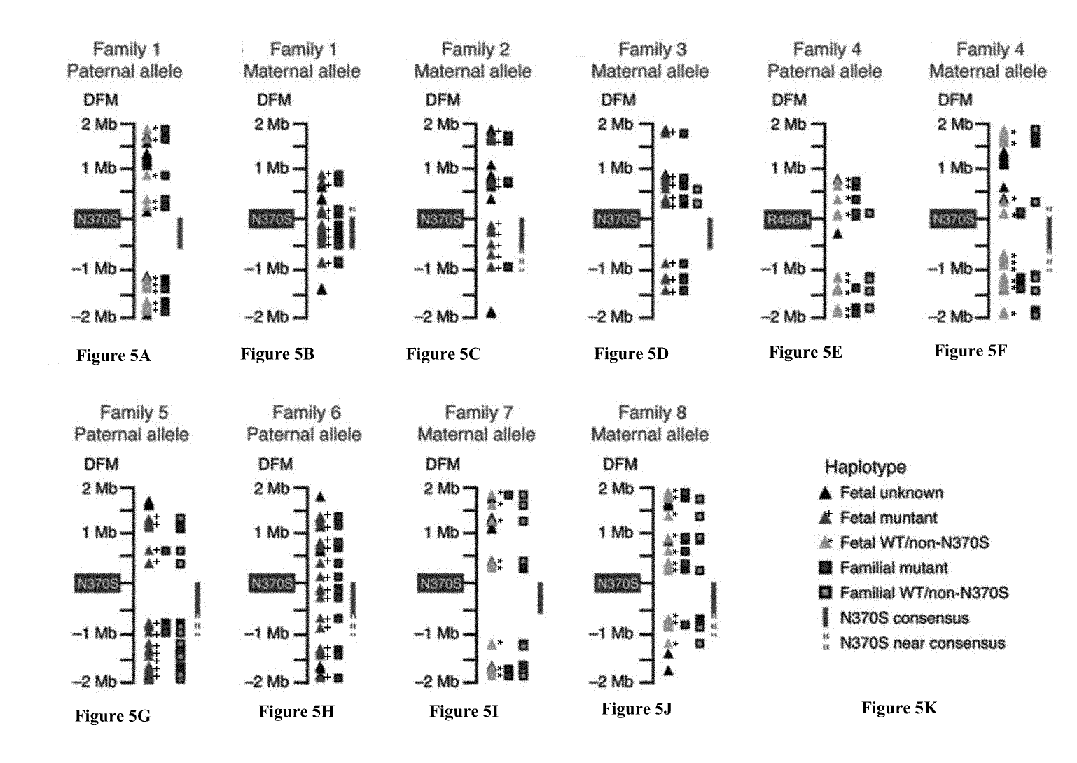

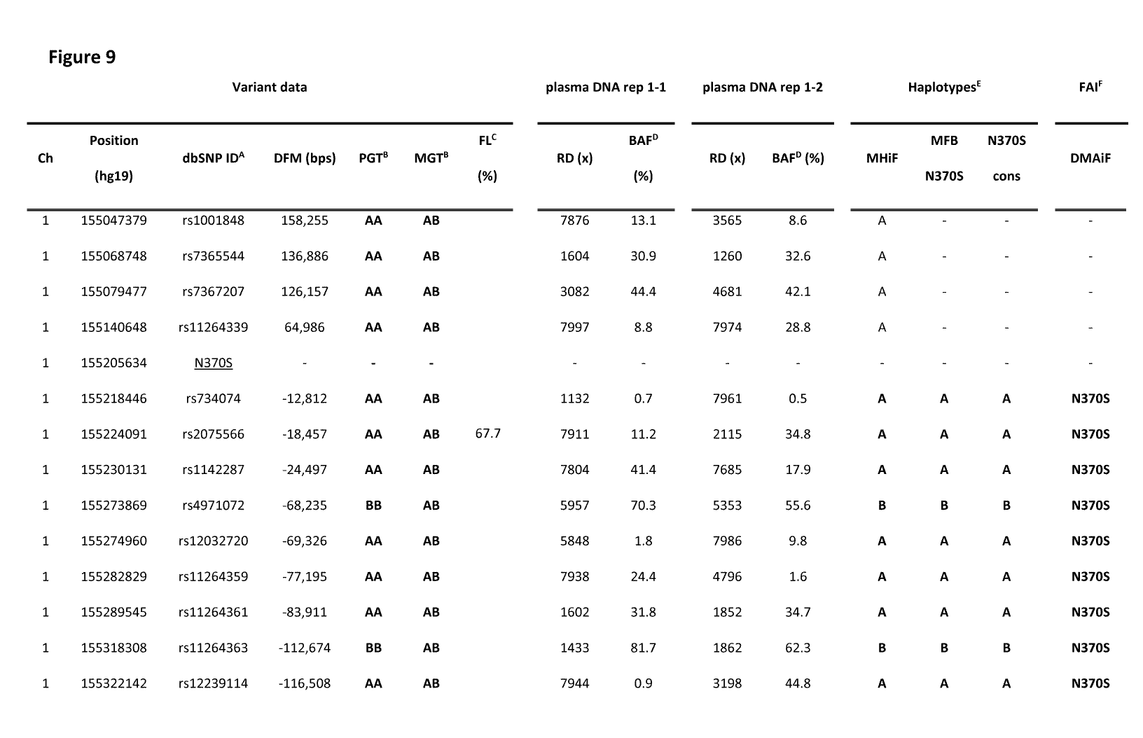

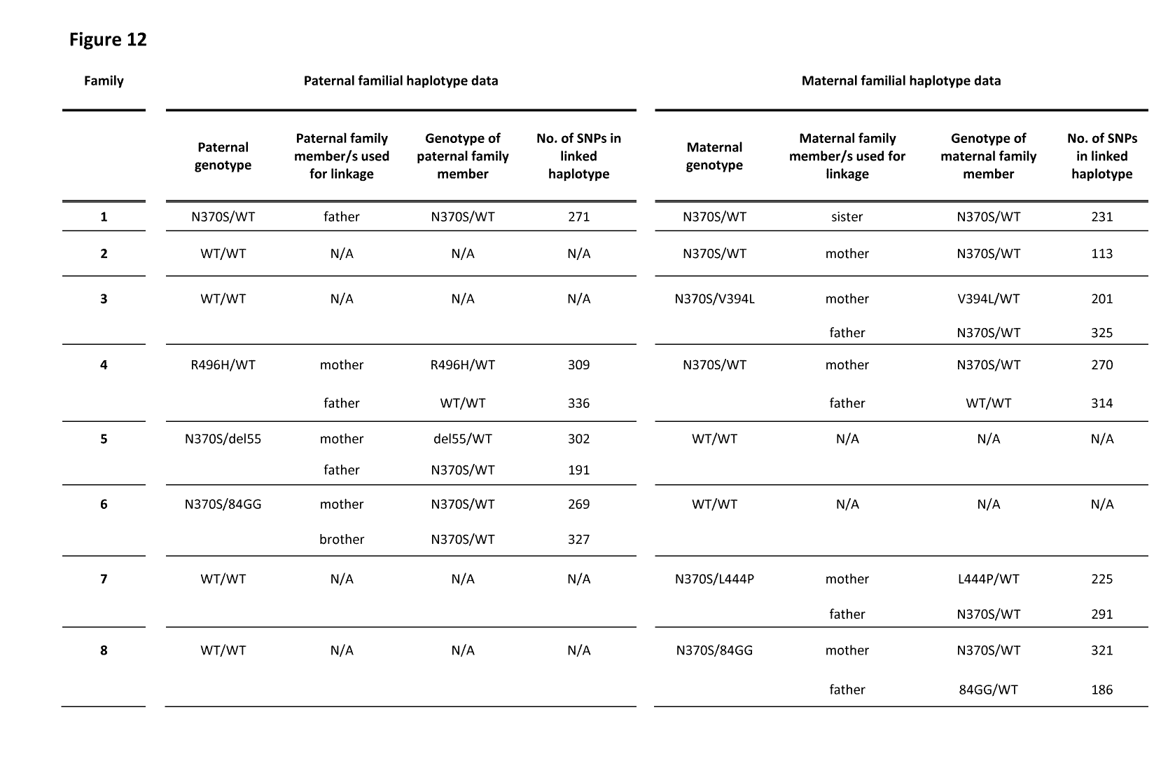

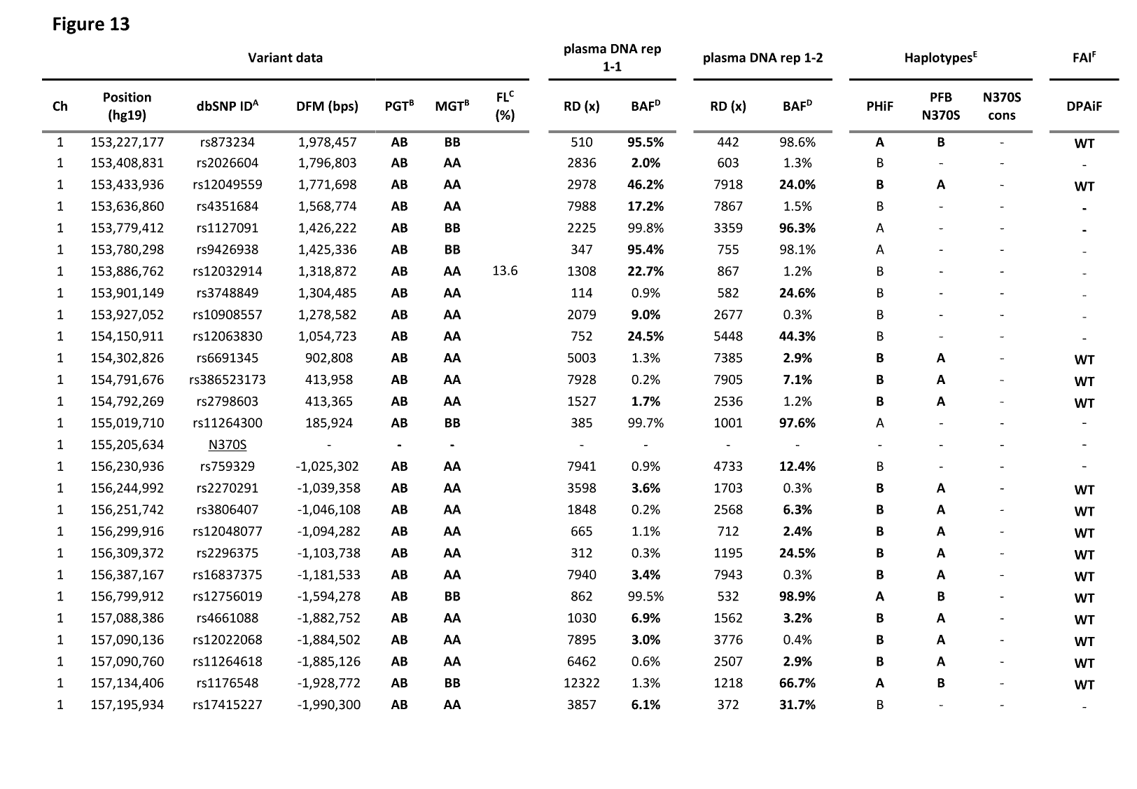

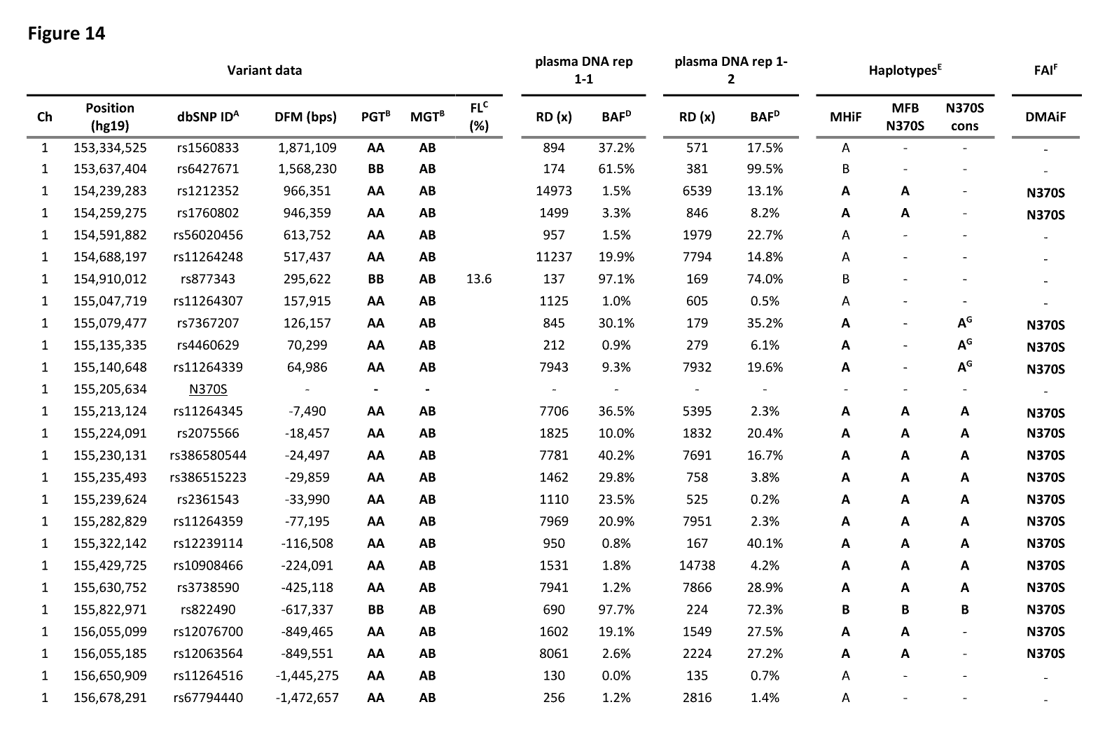

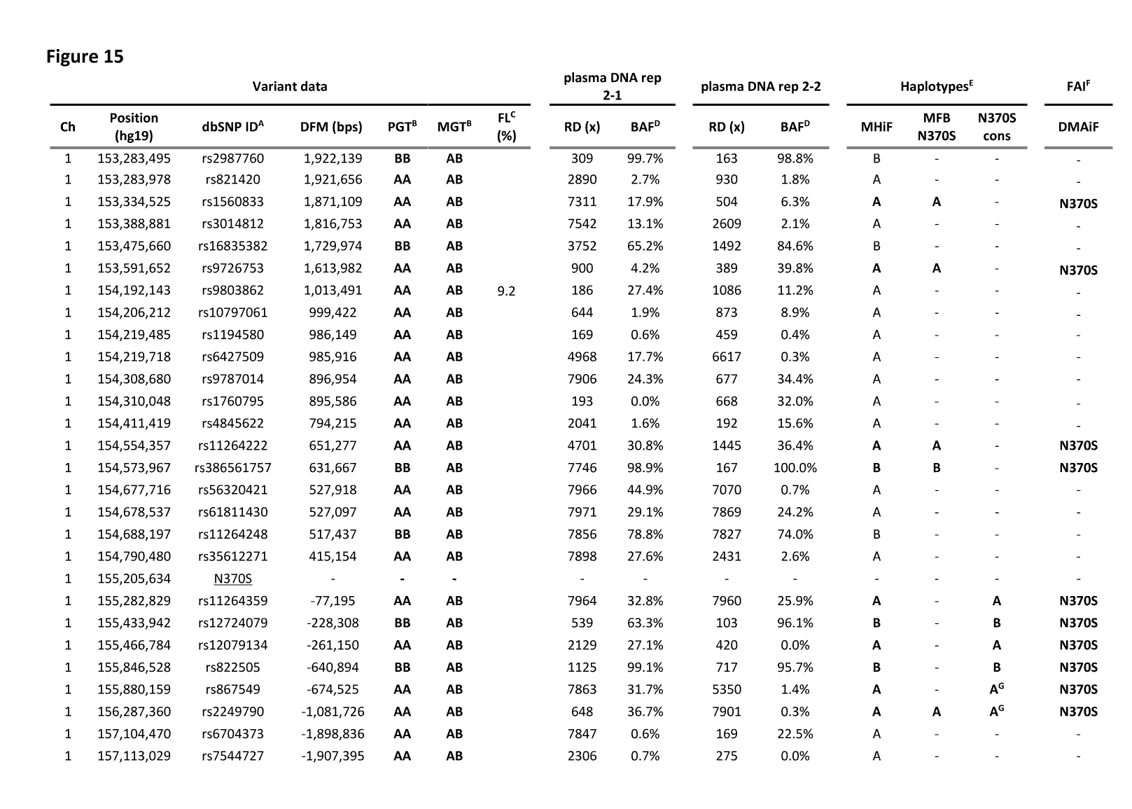

Having setup the framework with which to embark on streamlined NIPD for the N370S founder mutation, the inventors returned to families 1 and 2 and retested the same samples using the expanded sequencing panel. One of the primary issues with the previous analysis involving these families was the small size of linkage-based haplotypes in the family 1 paternal N370S allele and the family 2 maternal N370S allele (Table 4). As expected, the large sequencing panel clearly solved this issue for families 1 and 2 (and, essentially, all families in this study). Ranging from 113 to 336 phased SNPs, all parental family-based haplotypes in the current investigation were of substantial size and content to enable scoring of fetal haplotypes with generally high confidence (FIG. 12). Interestingly, the family 1 father turned out to be homozygous for the entire N370S consensus and near-consensus sequence by the new panel analysis. Nonetheless, his linkage-based N370S haplotype facilitated highly unambiguous identification of his WT allele in the fetus (FIG. 5A and FIG. 13). This test result was clearly of much higher quality, in terms of fetal haplotype size (14 phased SNPs), in comparison with the previous test (2 phased SNPs) involving the same samples (FIG. 3A). Regarding the family 1 maternal allele, there was little doubt from the previous panel whether the mother had transmitted her N370S allele to the fetus (9 phased SNPs in fetus; FIG. 3B and FIG. 9). In the newer panel, it was even more obvious that the mother had transmitted her N370S allele to the fetus based on clear matches between the fetal haplotype and the mother's family-based N370S allele as well as her consensus and near-consensus N370S sequences (17 phased SNPs altogether; FIG. 5B and FIG. 14). With family 2, the value and importance of the consensus N370S haplotype grew manifold after reanalysis on the larger sequencing panel. In the previous assessment, only 1 of 3 SNPs in the fetal haplotype was phased to the family-based N370S allele (FIG. 3C). In the newer evaluation, the fetal haplotype was much larger, but only 5 SNPs were phased to the family-based maternal N370S haplotype, one of which was isolated on the 3′ side of GBA (1 Mb distance from the mutation). To strengthen the certainty of this test result, the fetal haplotype was compared to the parent-specific consensus and near-consensus N370S haplotype. This comparison yielded another 5 phased SNPs located 3′ to the mutation, which, together with family-based fetal alleles, led to the correct diagnosis of the N370S mutation in the family 2 fetus (based on 10 phased SNPs altogether; FIG. 5C and FIG. 15).

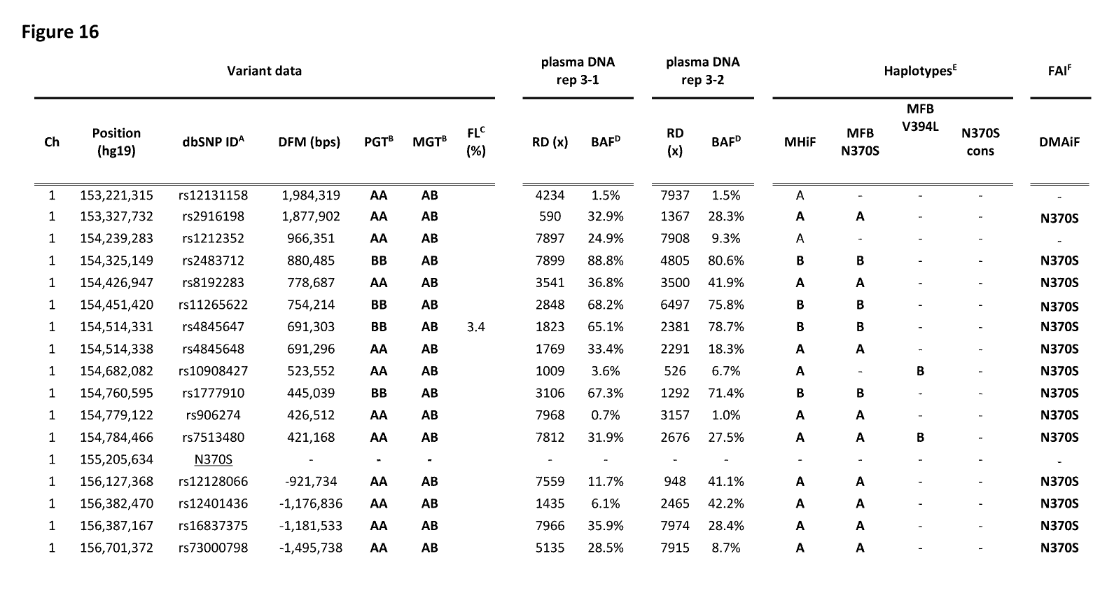

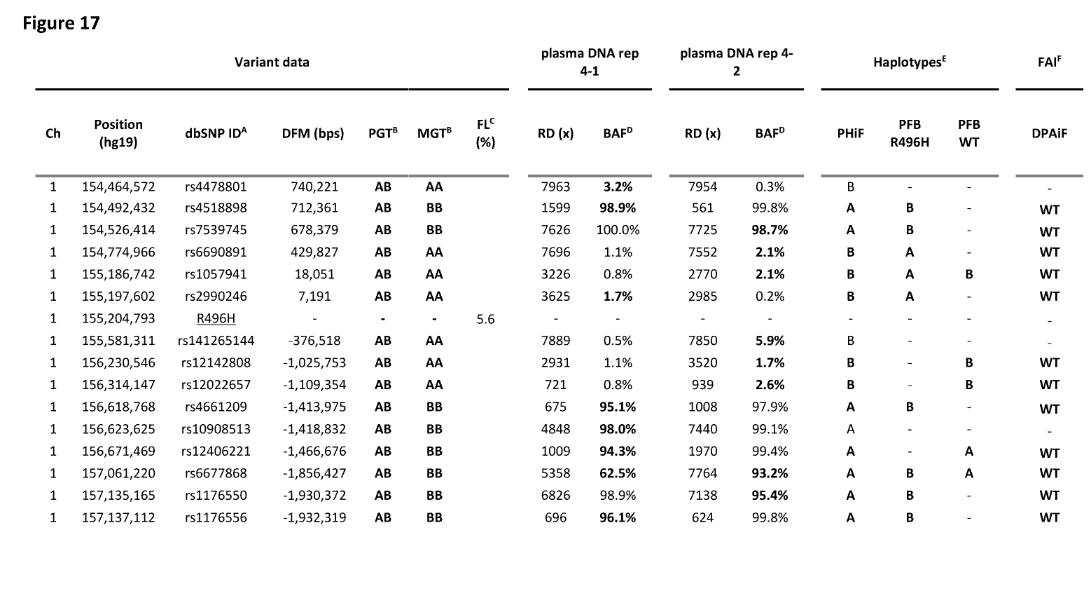

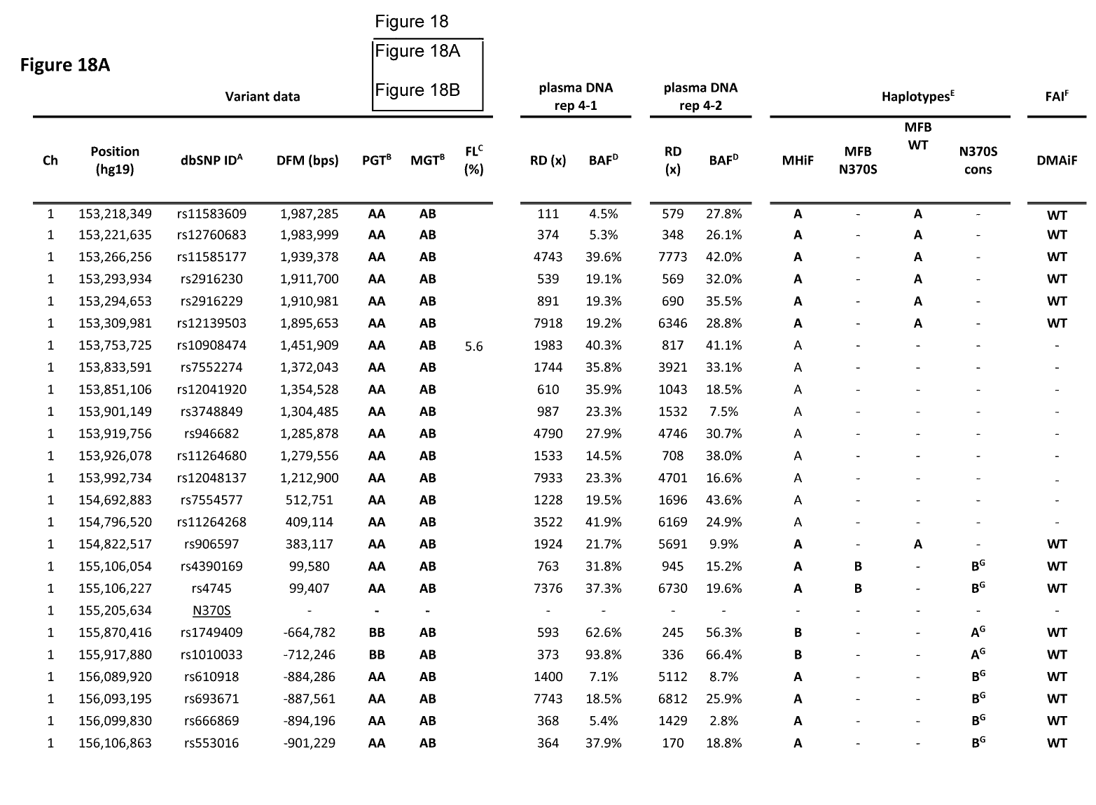

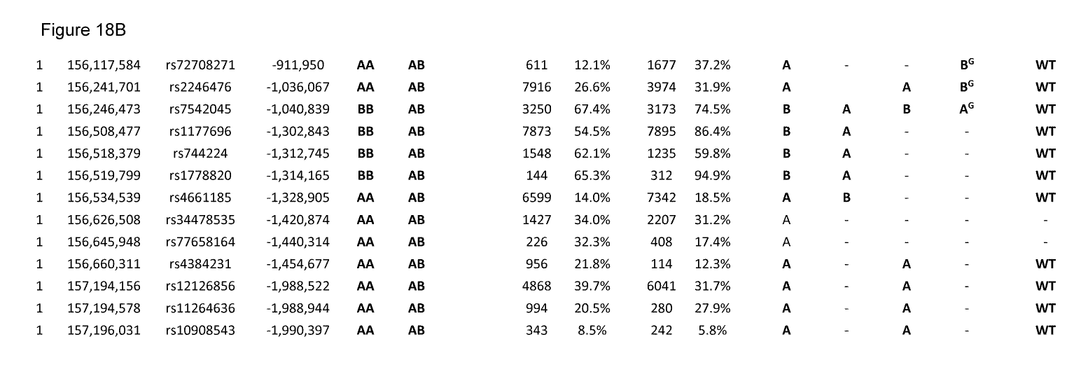

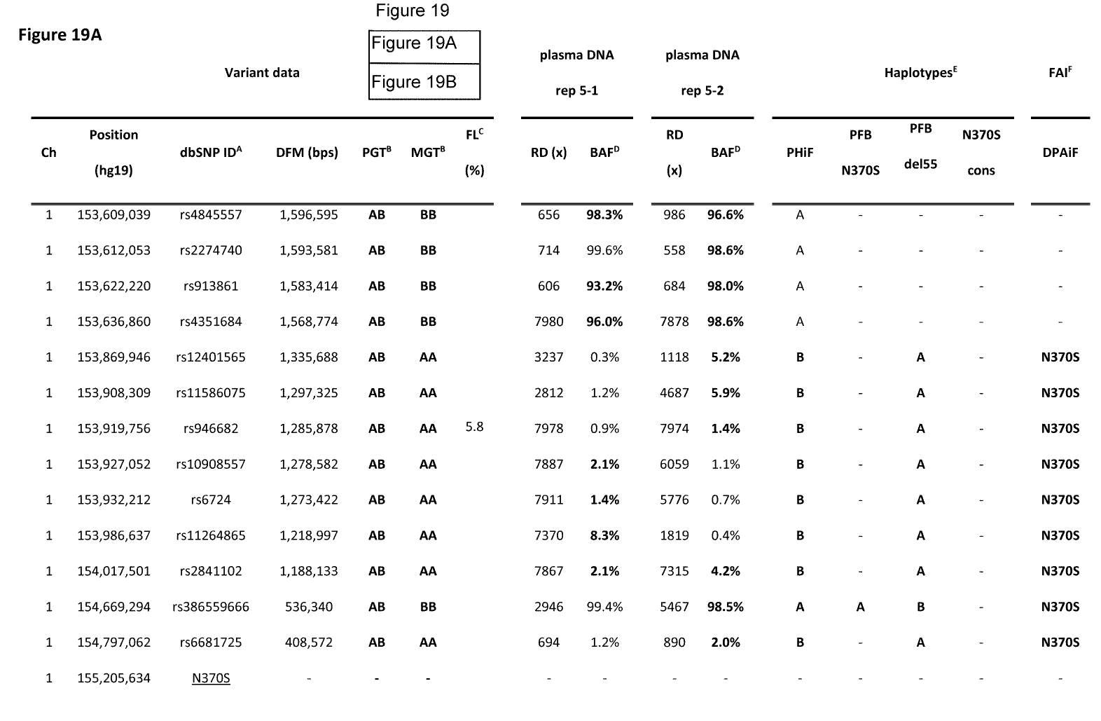

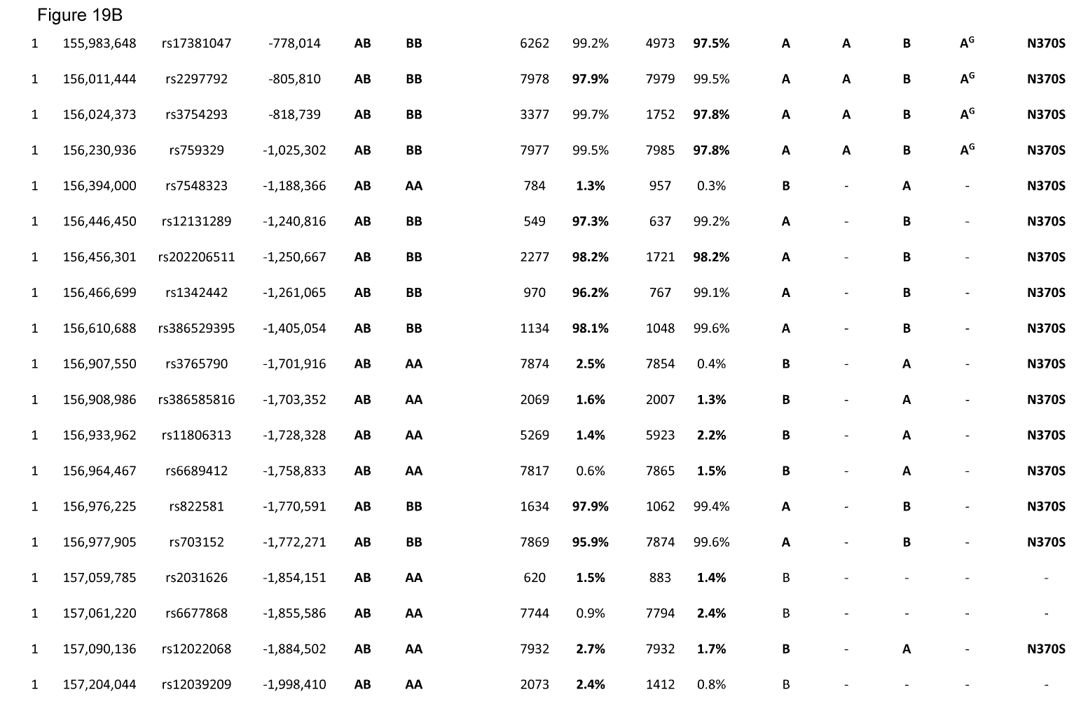

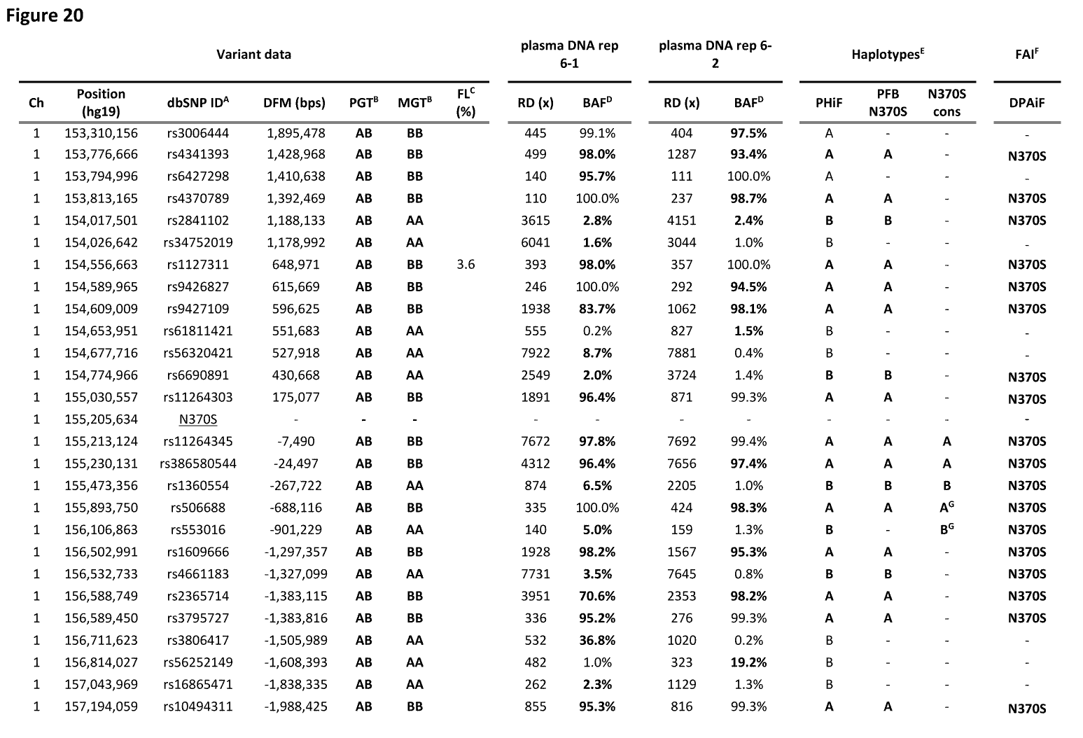

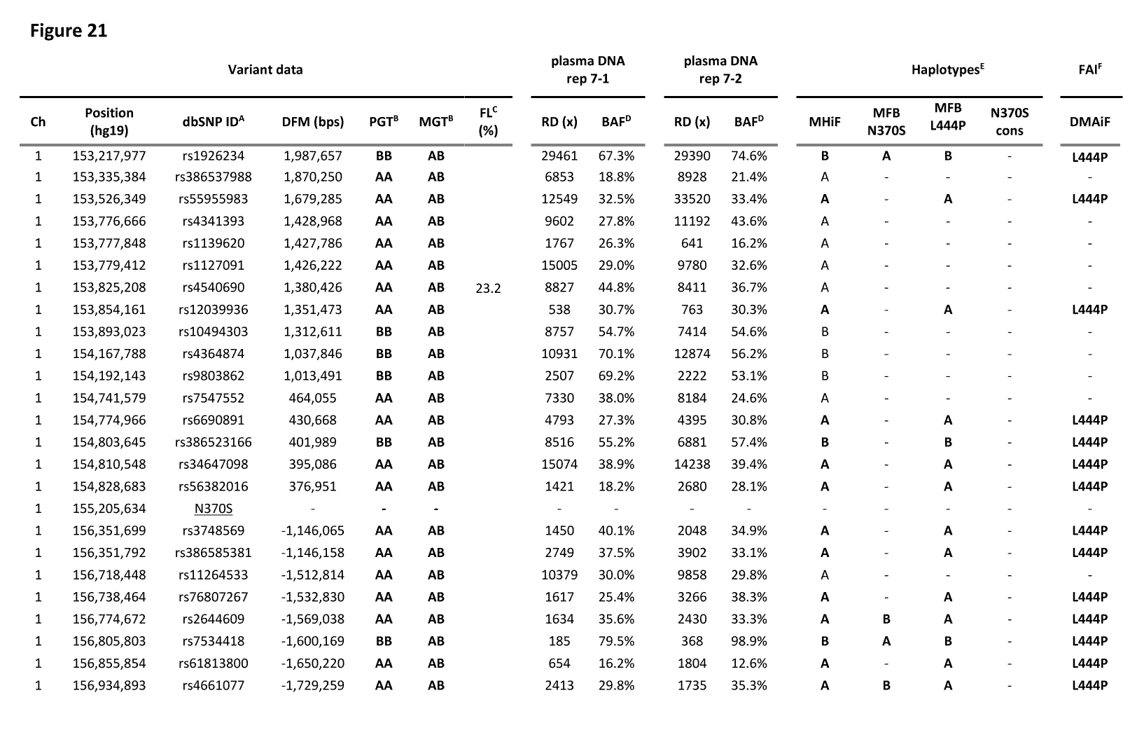

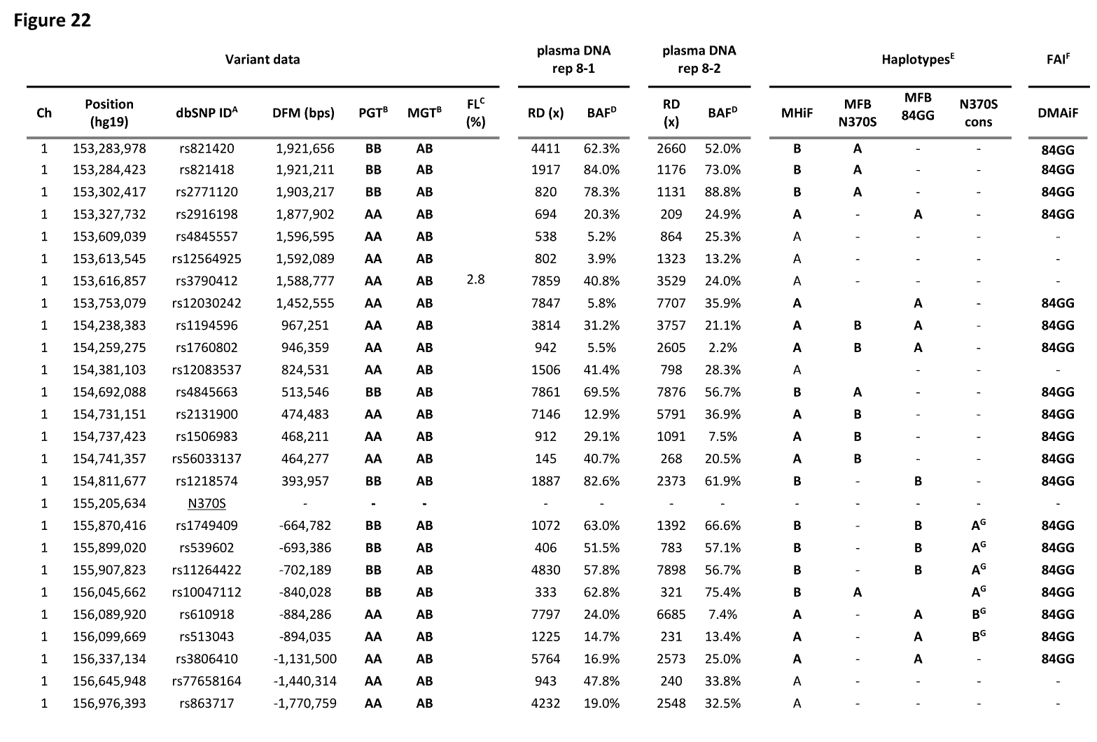

The principles set forth in these preliminary tests were subsequently put into practice for fetal allele identification involving families 3 through 8 (FIG. 5, D-J, and FIG. 16, FIG. 17, FIGS. 18A-B, FIGS. 19A-B, FIG. 20, FIG. 21, and FIG. 22). Of particular importance is the fact that 4 of 6 N370S-paired alleles in these families were typed noninvasively with the aid of the N370S consensus and/or near-consensus sequence (FIGS. 5, F-H, and J). These results thereby confirmed the assumption that the founder N370S haplotype is a valuable tool for incorporation into standard NIPD protocol. Another point to consider, which supports the use of larger sequencing panels in NIPD in general, is the fact that, even when a non-founder GBA mutation was tested (such as that of the family 4 paternal allele; FIG. 5E and FIG. 17), the extended sequencing panel facilitated construction of a well-defined fetal haplotype nonetheless.

To summarize, the outcomes of this proof-of-concept study are presented in FIG. 23. All noninvasive test results were validated with conventional prenatal or postnatal diagnostics.

### Example 2: Noninvasive Prenatal Diagnosis of Cystic Fibrosis

First, a consensus DelF508 founder haplotype is identified and constructed, such as by the methods disclosed hereinabove, inter alia by using the publicly available haplotype database, such as HapMap or deCode or whole genome sequencing data from one or more ethnicities.

Subsequently, peripheral blood samples are collected from pregnant female indices and plasma is separated from peripheral blood by methods known in the art, e.g., centrifugation at 1,900×g for 10 minutes at 4° C. The plasma supernatant is then re-centrifuged at 16,000×g for 10 minutes at 4° C. and 3 ml of the resulting supernatant was used for cell-free DNA extraction such as with the QIAamp Circulating Nucleic Acid kit (QIAGEN) according to the manufacturer's protocol. The maternal plasma DNA extracts are then pre-amplified, in duplicate, such as with the SurePlex Amplification System (Illumina) ahead of downstream processing.

Thereafter, the DNA extracts suspected of having the DelF508 founder mutation are amplified with standard or allele-specific amplification methods followed by sequencing. Indexed next generation sequencing libraries are prepared and normalized (e.g., Illumina) according to the manufacturer's protocol followed by 2×150 bp pair-end sequencing to a mean depth of at least 500× for genomic and plasma DNA samples, respectively. After sequencing runs, the data are aligned to target sequences on the human reference and genotyping data is extracted

Fetal diagnosis of cystic fibrosis is ultimately achieved after comparing the paternal and maternal cell-free fetal DNA (cffDNA) haplotypes with DelF508 consensus haplotype.

### Example 3: Noninvasive Prenatal Diagnosis of Beta-Thalassemia

First, a consensus for the G6V mutation in the HBB gene founder haplotype is identified and constructed, such as by the methods disclosed hereinabove, inter alia by using the publicly available haplotype database, such as HapMap or deCode or whole genome sequencing data from one or more ethnicities.

Subsequently, peripheral blood samples are collected from pregnant female indices and plasma is separated from peripheral blood by methods known in the art, e.g., centrifugation at 1,900×g for 10 minutes at 4° C. The plasma supernatant is then re-centrifuged at 16,000×g for 10 minutes at 4° C. and 3 ml of the resulting supernatant was used for cell-free DNA extraction such as with the QIAamp Circulating Nucleic Acid kit (QIAGEN) according to the manufacturer's protocol. The maternal plasma DNA extracts are then pre-amplified, in duplicate, such as with the SurePlex Amplification System (Illumina) ahead of downstream processing.

Thereafter, the DNA extracts suspected of having the G6V founder mutation are amplified with standard or allele-specific amplification methods followed by sequencing. Indexed next generation sequencing libraries are prepared and normalized (e.g., Illumina) according to the manufacturer's protocol followed by 2×150 bp pair-end sequencing to a mean depth of at least 500× for genomic and plasma DNA samples, respectively. After sequencing runs, the data are aligned to target sequences on the human reference and genotyping data is extracted

Fetal diagnosis of Beta-thalassemia is ultimately achieved after comparing the paternal and maternal cell-free fetal DNA (cffDNA) haplotypes with G6V consensus haplotype.

### Example 4: Noninvasive Prenatal Diagnosis of Bloom Syndrome

First, a consensus for the 736delATCTGAinsTAGATTC in the BLM gene founder haplotype is identified and constructed, such as by the methods disclosed hereinabove (e.g., using the HapMap or deCode or whole genome sequencing data from one or more ethnicities).

Subsequently, peripheral blood samples are collected from pregnant female indices and plasma is separated from peripheral blood by methods known in the art, e.g., centrifugation at 1,900×g for 10 minutes at 4° C. The plasma supernatant is then re-centrifuged at 16,000×g for 10 minutes at 4° C. and 3 ml of the resulting supernatant was used for cell-free DNA extraction such as with the QIAamp Circulating Nucleic Acid kit (QIAGEN) according to the manufacturer's protocol. The maternal plasma DNA extracts are then pre-amplified, in duplicate, such as with the SurePlex Amplification System (Illumina) ahead of downstream processing.

Thereafter, the DNA extracts suspected of having the 736delATCTGAinsTAGATTC founder mutation are amplified with standard or allele-specific amplification methods followed by sequencing. Indexed next generation sequencing libraries are prepared and normalized (e.g., Illumina) according to the manufacturer's protocol followed by 2×150 bp pair-end sequencing to a mean depth of at least 500× for genomic and plasma DNA samples, respectively. After sequencing runs, the data are aligned to target sequences on the human reference and genotyping data is extracted

Fetal diagnosis of Bloom syndrome is ultimately achieved after comparing the paternal and maternal cell-free fetal DNA (cffDNA) haplotypes with 736delATCTGAinsTAGATTC consensus haplotype.

### Example 5: Noninvasive Prenatal Diagnosis of Tay-Sachs

First, a consensus for the G269S mutationm in the HEXA gene founder haplotype is identified and constructed, such as by the methods disclosed hereinabove, inter alia by using the publicly available haplotype database, such as HapMap or deCode or whole genome sequencing data from one or more ethnicities.

Subsequently, peripheral blood samples are collected from pregnant female indices and plasma is separated from peripheral blood by methods known in the art, e.g., centrifugation at 1,900×g for 10 minutes at 4° C. The plasma supernatant is then re-centrifuged at 16,000×g for 10 minutes at 4° C. and 3 ml of the resulting supernatant was used for cell-free DNA extraction such as with the QIAamp Circulating Nucleic Acid kit (QIAGEN) according to the manufacturer's protocol. The maternal plasma DNA extracts are then pre-amplified, in duplicate, such as with the SurePlex Amplification System (Illumina) ahead of downstream processing.

Thereafter, the DNA extracts suspected of having the G269S founder mutation are amplified with standard or allele-specific amplification methods followed by sequencing. Indexed next generation sequencing libraries are prepared and normalized (e.g., Illumina) according to the manufacturer's protocol followed by 2×150 bp pair-end sequencing to a mean depth of at least 500× for genomic and plasma DNA samples, respectively. After sequencing runs, the data are aligned to target sequences on the human reference and genotyping data is extracted

Fetal diagnosis of Tay-Sachs is ultimately achieved after comparing the paternal and maternal cell-free fetal DNA (cffDNA) haplotypes with G269S consensus haplotype.

### Example 6: Noninvasive Prenatal Diagnosis of Alpha 1-Antitrypsin Deficiency

First, a consensus for the E342K mutation in the SERPINA gene founder haplotype is identified and constructed, such as by the methods disclosed hereinabove, inter alia by using the publicly available haplotype database, such as HapMap or deCode or whole genome sequencing data from one or more ethnicities.

Subsequently, peripheral blood samples are collected from pregnant female indices and plasma is separated from peripheral blood by methods known in the art, e.g., centrifugation at 1,900×g for 10 minutes at 4° C. The plasma supernatant is then re-centrifuged at 16,000×g for 10 minutes at 4° C. and 3 ml of the resulting supernatant was used for cell-free DNA extraction such as with the QIAamp Circulating Nucleic Acid kit (QIAGEN) according to the manufacturer's protocol. The maternal plasma DNA extracts are then pre-amplified, in duplicate, such as with the SurePlex Amplification System (Illumina) ahead of downstream processing.

Thereafter, the DNA extracts suspected of having the E342K founder mutation are amplified with standard or allele-specific amplification methods followed by sequencing. Indexed next generation sequencing libraries are prepared and normalized (e.g., Illumina) according to the manufacturer's protocol followed by 2×150 bp pair-end sequencing to a mean depth of at least 500× for genomic and plasma DNA samples, respectively. After sequencing runs, the data are aligned to target sequences on the human reference and genotyping data is extracted

Fetal diagnosis of alpha-1-antitrypsin deficiency was ultimately achieved after comparing the paternal and maternal cell-free fetal DNA (cffDNA) haplotypes with E342K consensus haplotype.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for non-invasively predicting an increased risk of disease-associated maternal and/or paternal haplotypes inherited by a fetus of a pregnant female, the method comprising:
(i) obtaining at least a replicate of a fetal nucleic acid sequence sequenced at a depth of at least 100× coverage, said fetal nucleic acid sequence being derived from a single DNA sample obtained from the pregnant female; and
(ii) analyzing said replicate of fetal nucleic acid sequence, wherein a high identity of said fetal haplotype to a consensus haplotype indicates that said fetus is a carrier of a maternal and/or paternal haplotype;
thereby predicting an increased risk of a disease-associated maternal and/or paternal haplotype inherited by said fetus.

2. A method for non-invasively predicting an increased risk of a monogenic disease or disorder in a fetus of a pregnant female, the method comprising:
(i) obtaining at least a replicate of a fetal nucleic acid sequence sequenced at a depth of at least 100× coverage, said fetal nucleic acid sequence being derived from a single DNA sample obtained from the pregnant female; and
(ii) analyzing said replicate of fetal nucleic acid sequence, wherein a high identity of said fetal haplotype to a consensus haplotype indicates that said fetus is a carrier of a maternal and/or paternal haplotype;
thereby predicting an increased risk of a monogenic disease or disorder in said fetus.

3. The method of claim 1, wherein said DNA is cell free fetal DNA (cffDNA).

4. The method of claim 1, wherein said sample is a plasma sample.

5. The method of claim 1, wherein said fetal nucleic acid sequence is sequenced at a depth of at least 2,500× mean coverage.

6. The method of claim 1, wherein said fetal nucleic acid sequence is sequenced at a depth of at least 3,000× mean coverage.

7. The method of claim 1, wherein said analyzing said fetal nucleic acid sequence comprises comparing said fetal haplotype to a consensus haplotype.

8. The method of claim 1, wherein said consensus haplotype is a population-based haplotype based on subjects unrelated to said fetus.

9. The method of claim 1, wherein said analyzing said replicate of fetal nucleic acid sequence comprises determining one or more paternal haplotype informative single-nucleotide polymorphism (SNP)s in at least one replicate of fetal nucleic acid, said paternal haplotype informative SNPs are not present in the maternal genotype, thereby determining unique paternal SNPs identified in the fetus.

10. The method of claim 1, wherein said analyzing said replicate of fetal nucleic acid sequence comprises determining maternal haplotype informative SNPs in fetal nucleic acid, thereby determining maternal haplotype in said fetus.

11. The method of claim 1, wherein said maternal haplotype comprises a founder haplotype encompassing a founder mutation, said method being useful for predicting an increased risk of said founder mutation in said fetus.

12. The method of claim 2, wherein said monogenic disease or disorder is caused by, or strongly associated with, a founder mutation.

13. The method of claim 2, wherein said monogenic disease or disorder presents with autosomal recessive inheritance.

14. The method of claim 2, wherein said monogenic disease or disorder is selected from the group consisting of Gaucher disease, cystic fibrosis, beta-thalassemia, sickle cell anemia, Alpha 1-antitrypsin deficiency, Bardet Biedl syndrome, Bloom syndrome, Canavan disease, Familial Dysautonomia, Fanconi anemia C, Hermansky-Pudlak syndrome, Joubert syndrome 2, Microcephaly with complex motor and sensory axonal neuropathy, Maple Syrup Urine Disease (MSUD), Mucolipidosis IV, Nemaline myopathy. Niemann-Pick disease A, Usher syndrome I, Usher syndrome III, Walker Warburg syndrome and Zelweger syndrome.

15. The method of claim 1 wherein said coverage is for a given single nucleotide polymorphism (SNP).
